(19) United States
(12) Patent Application Publication
(10) Pub. No.: US 2016/0136225 A1
(43) Pub. Date: May 19, 2016

(54) METHODS AND COMPOSITIONS FOR ENHANCING STEM CELL MOBILIZATION

(71) Applicant: STEMTECH INTERNATIONAL, INC., Pembroke Pines, FL (US)

(72) Inventors: Christian Drapeau, Austin, TX (US); Gitte S. Jensen, Port Dover (CA)

(73) Assignee: Stemtech International, Inc., Pembroke Pines, FL (US)

(21) Appl. No.: 14/900,032

(22) PCT Filed: Jun. 19, 2014
(86) PCT No.: PCT/US14/43237

§ 371 Date: Dec. 18, 2015

Related U.S. Application Data
(60) Provisional application No. 61/837,045, filed on Jun. 19, 2013.

(51) Int. Cl. A61K 36/886; A61K 9/48; A61K 36/815; A61K 36/70; A61K 36/068; A61K 36/06; A61K 31/715; A61K 9/00; A61K 35/20

(52) U.S. Cl. CPC A61K 36/886; A61K 9/0053; A61K 9/48; A61K 36/815; A61K 35/20; A61K 36/068; A61K 36/06; A61K 31/715; A61K 36/70

(57) ABSTRACT

The present invention provides a method of using mobilization agents to enhance stem cell mobilization in a subject, including hematopoietic stem cells (HSCs) and bone marrow stem cells (BMSCs). In one embodiment, a blended composition of fruits, mushrooms, microorganisms, maternal fluids, and extracts thereof are used to promote trafficking of stem cells, resulting in migration of the stem cells to specific sites of maintenance and repair within tissues and/or organs. Increased circulation of HSCs and/or BMSCs and migration towards sites of maintenance and the natural regeneration mechanisms in the body. Further provided is a dosing regimen for the administration of fucoidan and a method of enhancing release and circulation of stem cells.

### FIELD OF THE INVENTION

The present invention relates to methods and compositions for enhancing the mobilization of stem cells.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Stem cells (SC) are defined as cells with the unique capacity to self-replicate throughout the entire life of an organism and to differentiate into various cell types of the body. Two well-known types of stem cells are embryonic stem cells and adult stem cells. Embryonic stem cells (ESCs) are extracted from 5-10 day old embryos called blastulas. Once isolated, ESCs can be grown in vitro and led to differentiate into various types of tissue cell (such as heart cells, liver cells, nervous cells, and kidney cells), after which they can be injected in specific tissues in order to regenerate the tissue.

Adult stem cells (ASCs) are undifferentiated or primitive cells that can self-renew and differentiate into specialized cells of various tissues and are found in any living organism after birth. ASCs have been isolated from various tissues such as the liver (oval cells), the intestine (intestinal crypt stem cells), muscles (satellite cells), the brain (neural stem cells), and recently the pancreas (nestin positive pancreatic stem cells). Umbilical cord stem cells and placental stem cells are considered ASCs.

The role of ASCs found in tissues (tissue stem cells) is to maintain and repair the tissue in which they are found, although recent studies have reported that ASCs from one tissue may have the ability to develop into cell types characteristic of other tissues. For example, oval cells in the liver were shown in vitro to have the ability to become insulin-producing pancreatic cells. Nevertheless, the general view is that local stem cells are primarily involved in minor repair of the tissue in which they reside. In the case of significant injury or degeneration, the number of new tissue cells found in healing tissue far exceeds the capacity of local stem cells to duplicate and differentiate, suggesting that stem cells coming from other sites must be involved in the process of repair.

Although many tissues contain their own specific population of tissue stem cells, certain ASCs of key interest are those primarily found in the bone marrow and blood, Tissue stem cells are traditionally believed to be limited in their ability to differentiate into other tissues. However bone marrow stem cells (BMSC) were recently shown to have significant capability to become cells of other tissues.

It is difficult to freeze these processes in time to extract a cohesive, comprehensive portrait of regenerative mechanisms in the body. Nonetheless, enough information is available to affirm that different stem cells in the body, whether BMSCs, HSCs, marrow stromal cells (MSCs), multipotent adult progenitor cells (MAPCs), very small embryonic-like stem cells (VSEL), epiblast-like stem cell (ELSC) or blastomere-like stem cell (BLSC), constitute a broad component of the body's natural healing system. Since stem cells are capable of differentiating into a broad variety of cell types, they play an important role in the healing and regenerative processes of various tissues and organs. Bone marrow stem cells, including marrow stromal cells (MSCs), are released from tissues of origin, and circulate in a subject's circulatory or immune system to migrate into various organs and tissues to become mature, terminally differentiated cells. Therefore, enhancement of stem cell trafficking (i.e., release, circulation, homing and/or migration) can amplify these physiological processes and provide potential therapies for various pathologies. There are compositions and methods that utilize stem cell mobilization as a therapeutic approach. However, existing methods of promoting stem cell mobilization suffer from significant drawbacks, including poor kinetic performance, high cost, inconvenient methods of administration and unwanted side effects. One leading approach, injection of granulocyte colony-stimulating factor (G-CSF) or recombinant forms thereof, requires days to achieve peak circulating HSC numbers. The opposite problem exists with administration of interleukin-8 (IL-8), which acts only within minutes and has a short-lived effect on elevating circulating HSC levels in the bloodstream. G-CSF and a different molecule, CXCR4 antagonist AMD3100, can have significant side effects, including hemorrhaging, rupturing of the spleen, bloody sputum, bone disorders, among others. Thus, there is a need in the art for an effective and convenient method for delivering stem cell mobilization agents to human subjects, to obtain positive clinical benefits without side effects and at a reduced cost.

Accordingly, the inventive compositions and methods disclosed herein enhance the release, circulation, homing and/or migration of stem cells within the body to promote healing and treatment of damaged tissues, as well as aid in the regeneration of tissues that suffer from some level of cellular loss, for greater vitality and reduced incidence of disease.

### SUMMARY OF THE INVENTION

The following embodiments and aspects thereof are described and illustrated in conjunction with compositions and methods that are meant to be exemplary and illustrative, not limiting in scope. In one embodiment, the invention includes a method of increasing stem cell mobilization in a subject, including: providing a mobilization agent capable of increasing stem cell mobilization, and administering a quantity of the mobilization agent to the subject in an amount sufficient to increase stem cell mobilization in the subject. In another embodiment, the mobilization agent is a composition including one or more of the following components selected from the group including: Aloe or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In another embodiment, the mobilization agent is fucoidan. In another embodiment, the fucoidan is extracted from Undaria pinnatifida. In another embodiment, the quantity of the fucoidan is 250 mg. In another embodiment, the stem cell is a bone marrow-derived stem cell (BMSC). In another embodiment, the stem cell is a hematopoietic stem cell (HSC). In another embodiment, administering the quantity includes oral administration. In another embodiment, the oral administration includes use of a capsule or a pill. In other embodiments, oral administration is more than once a day. In other embodiments, oral administration is daily. In other embodiments, the capsule or a pill includes a quantity of about 50, 100, 150, 200, 250 mg or less of the one or mobilization agents. In other embodiments, includes a quantity of about 250, 500, 750, or 1000 mg or less of the one or more mobilization agents. In other embodiments, the capsule or a pill includes 750 mg or less of Aloe macroclada. In various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In various embodiments, the methods results in trafficking of stem cells following administration of the mobilization agent. In one embodiment, providing a mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration. In various embodiments, release of stem cells into the circulation from about 1, 2, or 3 hours following administration. In another embodiment, released stem cells enter the circulatory system and increase the number of circulating stem cells within the subject's body. In another embodiment, the percentage increase in the number of circulating stem cells compared to a normal baseline may about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

Another embodiment of the present invention provides a pharmaceutical composition including one or more of the following components selected from the group consisting of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof, and a pharmaceutically acceptable carrier. In various embodiments, the pharmaceutical composition includes Aloe. In various embodiments, the Aloe is Aloe macroclada. In various embodiments, the pharmaceutical composition includes a quantity of about 50, 100, 150, 200, 250 mg or less of the one or more components. In various embodiments, the pharmaceutical composition includes a quantity of about 250, 500, 750, or 1000 mg or less of the one or more components. In various embodiments, the pharmaceutical composition includes 750 mg or less of Aloe macroclada. In various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts mobilization and migration of endogenous stem cells in accordance with various embodiments of the present invention. Under normal physiological conditions or in response to disease or injury, hematopoietic stem cells mobilize from compartments such as bone (A) and circulate into the bloodstream (B), migrate towards tissues to promote repair and regeneration in different parts of the body (C).

FIG. 2 shows a schematic illustration of the steps involved in the migration of a stem cell, underscoring the role of CXCR4, in accordance with an embodiment of the present invention.

FIG. 3 provides graphs illustrating a typical time course of stem cell migration in the human body after consumption of (A) whole Lycium barbarum (LB) fruit and (B) colostrum (Col), in accordance with various embodiments of the present invention. For both products, the thin lines show individual responses. For LB, the thick dotted line is the average response while the thick line shows the time course of the response with the average peak response at 45 minutes. For Col, all participants peaked at 60 minutes, so the thick lines show the average time course of the response.

FIG. 4 provides a graph illustrating a typical time course of stem cell migration in the human body after consumption of (A) a polysaccharide rich fraction of mushroom (Cordyceps sinensis, Ganoderma lucidum, Hericium erinaceus), and (B) spirulina or an extract thereof, in accordance with an embodiment of the present invention.

FIGS. 5A, 5B and 5C are flow cytometry profiles of blood samples showing the proportions of CD34+ lymphocytes from the peripheral blood of a human volunteer after ingestion of L. barbarum, colostrum and mushroom polysaccharides, respectively, in accordance with an embodiment of the present invention. The X axis displays fluorescence intensity of the stem cell marker. The M1 marker indicates events showing positivity for the stem cell marker CD34.

FIG. 6 is a graph illustrating the expression of CXCR4 molecules on the surface of CD34+ circulating stem cells before and after consumption of LB, Col, and mushroom polysaccharides, in accordance with an embodiment of the present invention.

FIG. 7 provides a graph illustrating a typical time course of stem cell migration in the human body after consumption of Lycium barbarum, colostrum, spirulina and a polysaccharide rich fraction of mushroom (Cordyceps sinensis, Ganoderma lucidum, Hericium erinaceus), in accordance with an embodiment of the present invention.

FIG. 8 depicts changes in circulating CD34+ hematopoietic stem cells in human volunteers following oral administration of fucoidan extracted from Undaria pinnatifida in accordance with various embodiments of the present invention. Baseline levels of peripheral blood stem cells were quantified in volunteers. Volunteers then ingested 250 mg of fucoidan extracted from Undaria pinnatifida. The levels of stem cells were subsequently measured at 45, 90 and 180 minutes. The number of circulating stem cells increased on average by 17%, 23% (P<0.02) and 32% (P<0.02), respectively.

FIG. 9 depicts the results of consuming fucoidan from algae species, Chordaria cladosiphon in accordance with various embodiments of the present invention. Consumption of 250 mg of fucoidan from Chordaria cladosiphon gave an average decrease in the number of circulating stem cells under the same conditions.

FIG. 10 depicts the results of consuming a combination of Polygonum multiflorum, blue-green algae, and fucoidan in accordance with various embodiments of the present invention. Consumption of the combination including Polygonum multiflorum resulted in a transitory increase in the number of circulating stem cells compared to placebo under the same conditions. The combination containing Polygonum multiflorum was shown to trigger a modest increase in the number of circulating stem cells by 13±6% (n=7) (p<0.05). The increase exceeded 25% in 2 of the participants.

FIG. 11 depicts changes in circulating CD34+ hematopoietic stem cells in human volunteers following oral administration extracts from Aloe macroclada in accordance with various embodiments of the present invention. The levels of stem cells were subsequently measured at 60, 120, 180 and 240 minutes as shown, with a rapid increasing rate of over 60 to 120 minute time points, sustained through subsequent measurements at 180 and 240 minutes.

FIG. 12 depicts changes in circulating CD45^{dim} CD34⁺. hematopoietic stem cells in human volunteers following oral administration extracts from Aloe macroclada in accordance with various embodiments of the present invention when compared to indigenous pellet forms. Indigenous pellets (340 mg) did not have any effect on CD45^{dim} CD34⁺ cells. However, 250 mg and 750 mg of sap/gel triggered an increase in the number of circulating CD45^{dim} CD34⁺ cells that reached 27% and 32% at 120 minutes, though the effect did not reach significance. Results seen with 250 mg and 750 mg of the sap/gel did not show any significant difference and were therefore pooled together. When pooled the data with the sap/gel (n=8) revealed a 29.6% increase (p<0.02) in the number of circulating CD45^{dim} CD34⁺ cells at 120 minutes.

FIG. 13 depicts changes in circulating CD34+ hematopoietic stem cells in human volunteers following oral administration of indigenous pellets. Indigenous pellets (340 mg) triggered an 18% increase in the number of circulating CD34⁺ cells, though this did not reach significance. Doses of 250 mg and 750 mg of sap/gel triggered an increase in the number of circulating CD34⁺ cells that reached 29.8% and 32% at 120 minutes. However, only the effect seen with 250 mg reached significance (p<0.04). Results seen with 250 mg and 750 mg of the sap/gel did not show any significant difference and were therefore pooled together. Data with the sap/gel (n=8) revealed a 29.9% increase (p<0.001) in the number of circulating CD34⁺ cells at 120 minutes.

FIG. 14 depicts changes in circulating CD34⁺ KDR⁻ hematopoietic stem cells in human volunteers following oral administration extracts from Aloe macroclada in accordance with various embodiments of the present invention when compared to indigenous pellet forms. Indigenous pellets (340 mg) triggered a 21.9% increase in the number of circulating CD34⁺ KDR⁻ cells at 120 minutes (p<0.03). Doses of 250 mg triggered an increase in the number of circulating CD34⁺ KDR⁻ cells of 42.4% at 120 and 22% at 180 minutes, though the effect did not reach significance. Doses of 750 mg triggered an increase in the number of circulating CD34⁺ KDR⁻ cells of 47.2% at 120 and 27.2% at 180 minutes, though the effect also did not reach significance. Results seen with 250 mg and 750 mg of the sap/gel did not show any significant difference and were therefore pooled together. All data with the sap/gel (n=8) revealed a significant 44.8% increase in the number of circulating CD34⁺ cells at 120 minutes (p<0.01) and 24.7% at 180 minutes (p<0.02).

FIG. 15 depicts changes in circulating CD45⁻ CD31⁺ KDR⁺. hematopoietic stem cells in human volunteers following oral administration extracts from Aloe macroclada in accordance with various embodiments of the present invention when compared to indigenous pellet forms. Indigenous pellets (340 mg) triggered an 80.6% and 69% increase in the number of circulating CD45⁻ CD31⁺ KDR⁺ cells at 120 (p<0.02) and 180 minutes (p<0.03), respectively. Doses of 250 mg triggered an increase in the number of circulating CD45⁻ CD31⁺ KDR⁺ cells of 32.4% and 46.8% at 120 and 180 minutes, respectively, though only the effect at 180 minutes reach significance (p<0.003). Doses of 750 mg triggered a significant increase in the number of circulating CD45⁻ CD31⁺ KDR⁺ cells of 75.4% at 180 (p<0.02). Results seen with 250 mg and 750 mg of the sap/gel did not show any significant difference and were therefore pooled together. All data with the sap/gel (n=8) revealed a 61.1% increase (p<0.0004) in the number of circulating CD34⁺ cells at 180 minutes.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul., 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods described herein. For purposes of the present invention, the following terms are defined below.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Aloe” as used herein refers to all species of the genus Aloe. Examples of Aloe species include Aloe barbadensis, A. africana, A. albida, A. albiflora, A. arborescens, A. argenticauda, A. aristata, A. ausana, A. bainesii, A. ballii, A. camperi, A. capitata, A. ciliaris, A. commixta, A. corallina, A. dewinteri, A. dichotoma, A. dinteri, A. eminens, A. erinacea, A. excelsa, A. ferox, A. forbesii, A. gracilis, A. haemanthifolia, A. helenae, A. hereroensis, A. indica, A. inermis, A. inyangensis, A. juddii, A. kilifiensis, A. khamiesensis, A. maculata, A. macroclada A. marlothii, A. namibensis, A. nyeriensis, A. peglerae, A. perfoliata, A. plicatilis, A. polyphylla, A. saponaria, A. scobinifolia, A. sinkatana, A. sladeniana, A. squarrosa, A. striatula, A. succotrina, A. suzannae, A. tenuior, A. thraskii, A. variegata, A. vera, A. viridiflora, A. vulgaris, A. wildii, in addition to the about 400 species within the genus Aloe.

“Colostrum” as used herein refers to a fluid secreted by the mammary glands of female mammals during the first few days of lactation, containing various nutrients and protease inhibitors that keep it from being destroyed by the processes of digestion. Humans produce relatively small amounts of colostrum in the first two days after giving birth, but cows produce about nine gallons of colostrum. Colostrum contains concentrated levels of important immune modulators, including Transfer Factor, PRP, IGF-1, n-acetyl neuraminic acid, GMP, nucleic acid and defensins. Colostrum extracts have been shown to activate phagocytosis by monocytes and increase the reactive oxygen burst in polymorph nucleated cells. Colostrum was also shown to trigger natural killer (NK) cell activation and also trigger the secretion of anti-inflammatory cytokines in in vitro cell-based assays. References herein to colostrum also include derivatives and artificial substitutes thereof.

“Differentiation” as used herein refers to the process by which cells become more specialized to perform biological functions. For example, hematopoietic stem cells, hematopoietic progenitors and/or stem cells may change from multipotent stem cells into cells committed to a specific lineage and/or cells having characteristic functions, such as mature somatic cells. Differentiation is a property that is often totally or partially lost by cells that have undergone malignant transformation.

“Enhancement,” “enhance” or “enhancing” as used herein refers to an improvement in the performance of or other physiologically beneficial increase in a particular parameter of a cell or organism. At times, enhancement of a phenomenon is quantified as a decrease in the measurements of a specific parameter. For example, migration of stem cells may be measured as a reduction in the number of stem cells circulating in the circulatory system, but this nonetheless may represent an enhancement in the migration of these cells to areas of the body where they may perform or facilitate a beneficial physiologic result, including, but not limited to, differentiating into cells that replace or correct lost or damaged function. In one embodiment, enhancement refers to a 15%, 20%, 30% or greater than 50% reduction in the number of circulating stem cells. In one specific, non-limiting example, enhancement of stem cell migration may result in or be measured by a decrease in a population of the cells of a non-hematopoietic lineage, such as a 15%, 20%, 30%, 50%, 75% or greater decrease in the population of cells or the response of the population of cells. In one embodiment, an enhanced parameter is the trafficking of stem cells. In one embodiment, the enhanced parameter is the release of stem cells from a tissue of origin. In one embodiment, an enhanced parameter is the migration of stem cells. In another embodiment, the parameter is the differentiation of stem cells. In yet another embodiment, the parameter is the homing of stem cells.

“Fucoidan” as used herein describes sulfated fucans obtained from algae. Fucoidan has been obtained from a broad range Algae species as provided in the following non-exhaustive list: Cladosiphon okamuranus, Chordaria flagelliformis, Ch. Gracilis, Saundersella simplex, Desmaestia intermedia, Dictyosiphon foeniculaceus, Dictyota dichotoma, Padina pavonica, Spatoglussum, schroederi, Adernocystis utricularis, Pylayella littoralis, Ascophyllum nodosum, Bifurcaria bifurcata, Fucus. Visculosus, F. spiralis, F. serratus, F. evaescens, Himanthalia lorea, Hizikia fusiforme, Pelvetia canaliculata, P. wrightii, Sargassum stenophyllum, S. honeri, S. Khellmanium, S. muticum, Alaria fistulosa, A. marginata, Arthrothammus bifidus, Chorda film, EckIonia kurome, E. cava, Eisenia bicyclis, Laminaria angustata, L. brasiliensis, L. cloustoni, L. digitata, L. japonica, L. religiosia, L. saccharina, Macrocystis integrifolia, M. pyrifera, Nereocystis luetkeana, Undaria pinnatifida, Petalonia fascia, Scytosiphon lomentaria. Substantial pharmaceutical research has been done on fucoidan, focusing primarily on two distinct forms: F-fucoidan, which is >95% composed of sulfated esters of fucose, and U-fucoidan, which is approximately 20% glucuronic acid, each of which is included in the term “fucoidan” as used herein. Depending on the source of the fucoidan, fucoidan can serve as a releasing agent in certain embodiments, while in other embodiments, fucoidan can serve as a migration agent.

“Hematopoietic agent” as used herein refers to a compound, antibody, nucleic acid molecule, protein, cell or other molecule that affects hematopoiesis. A molecular agent can be a naturally-occurring molecule or a synthetic molecule. In some instances, the agent affects the growth, proliferation, maturation, migration or differentiation or release of hematopoietic cells.

“Hematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. The term “hematopoietic progenitors”, “progenitor cells” or “blast cells” are used interchangeably in the present invention and describe maturing HSCs with reduced differentiation potential, but are still capable of maturing into different cells of a specific lineage, such as myeloid or lymphoid lineage. “Hematopoietic progenitors” include erythroid burst forming units, granulocyte, erythroid, macrophage, megakaryocyte colony forming units, granulocyte, erythroid, macrophage, and granulocyte macrophage colony-forming units.

“Homing” as used herein refers to the process of a cell migrating from the circulatory system into a tissue or organ. In some instances, homing is accomplished via tissue-specific adhesion molecules and adhesion processes. Homing may refer to the migration back to the bone marrow.

“Isolated biological component” (such as a nucleic acid molecule, polypeptide, polysaccharide or other biological molecule) as used herein refers to a biological component that has been substantially separated or purified away from other biological components in which the component naturally occurs. Nucleic acids and proteins may be isolated by standard purification methods, recombinant expression in a host cell, or chemically synthesized.

“Lycium barbarum” or “L. barbarum” as used herein refers to a small bright orange-red, ellipsoid berry or fruit grown. One exemplary source is in the north of China, primarily in the Ningxia Hui Autonomous Region. It is sometimes referred to as goji berry or wolfberry. L. barbarum belongs to the Solanaceae family, the nightshade family that includes hundreds of plant foods like potato, tomato, eggplant, and peppers (paprika). As used herein, L. barbarum and extracts thereof, refers to any fraction, extract, or isolated or purified molecule from L. barbarum. For example, the component is a protein or nucleic acid or a polysaccharide, a phytochemical, or a fraction of L. barbarum. Thus, in certain embodiments of the invention, components of L. barbarum are obtained by disrupting L. barbarum, adding an inorganic or organic solvent, and collecting fractions. Specific, non-limiting examples of fractions are isolated using high performance liquid chromatography, thin layer chromatography, or distillation. Fractionation may be based on the molecular weight or the hydrophobicity of the components of L. barbarum.

“Modulation” or “modulates” or “modulating” as used herein refers to upregulation (i.e., activation or stimulation), down regulation (i.e., inhibition or suppression) of a response or the two in combination or apart.

“Migration” as used herein refers to the central process for movement of cells in the development and maintenance of multicellular organisms. Cells often migrate in response to, and towards, specific external signals, commonly referred to as chemotaxis. Migration includes the process of a cell moving from the circulatory system into a tissue or organ. More specifically, circulating stem cells are tethered to the surface of capillary endothelium via expression of adhesion molecules of cell surfaces, resulting in cytoskeletal changes in both endothelium and stem cells, and allowing movement through the capillary wall en route to a tissue and/or organ site. In some instances, homing is accomplished via tissue-specific adhesion molecules and adhesion processes.

“Migration agent” as used herein are mobilization agents capable of promoting the process of a cell moving from the circulatory system into a tissue or organ. Migration of stem cells may be demonstrated, for example, by a decrease in circulating stem cells in the circulatory or immune system, or by the expression of surface markers and/or adhesion molecules on cell surfaces, which relate to homing, tethering, and/or extravasation of circulating stem cells to the surface of vessels such as capillary endothelium. Examples of migration agents include isolated or purified components extracted from Lycium barbarum, including a polysaccharide-rich fraction (fraction A) of Lycium barbarum extract, colostrum, including a protein-rich fraction (fraction B) of colostrum extract, fucoidan, including an isolated component or compound extracted from an algae, such as a compound found in a polysaccharide-rich fraction (fraction C) of algae extracts, including Chordaria cladosiphon, or other algaes, or extracts thereof, mushrooms, including an isolated component or compound extracted from a mushroom, such as a compound found in a polysaccharide-rich fraction (fraction D) of mushroom extracts, including Cordyceps sinensis or an extract thereof, Ganoderma lucidum or an extract thereof, Hericium erinaceus or an extract thereof, spirulina, including Arthrospira platensis, Arthrospira maxima, or extracts thereof. In different embodiments, this agent affects the migration of stem cells, such as CD34^{high} (CD34+) cells. In one embodiment, the migration agent decreases the number of bone marrow-derived stem cells and/or hematopoietic stem cells circulating in the peripheral blood. In another embodiment, the migration agent relates to enhanced expression of CXCR4 on circulating stem cells.

“Mushroom polysaccharides” as used herein refers to glucans found mainly in various species of mushrooms such as Cordyceps sinesis, Hercicium erinaceous, and Ganoderma lucidum. This also includes the numerous bioactive polysaccharides or polysaccharide-protein complexes from medicinal mushrooms that may enhance innate and cell-mediated immune responses, and exhibit antitumor activities in animals and humans.

“Pharmaceutically acceptable carriers” as used herein refer to conventional pharmaceutically acceptable carriers useful in this invention.

“Polysaccharide” as used herein refers to a polymer of more than about ten monosaccharide residues linked glycosidically in branched or unbranched chains.

“Progenitor cell” as used herein refers to a cell that gives rise to progeny in a defined cell lineage.

“Recruitment” of a stem cell as used herein refers to a process whereby a stem cell in the circulatory system migrates into specific site within a tissue or organ. Recruitment may be facilitated by a compound or molecule, such as a chemoattractant signal or cell receptor. For example, both CXCR4 and SDF-1 have identified roles in stem cell homing and migration.

“Releasing agent” as used herein are mobilization agents capable of promoting the release and egress of stem cells from a tissue of origin. Release of stem cells from a tissue of origin may be demonstrated, for example, by an increase in circulating stem cells in the circulatory or immune system, or by the expression of markers related to egress of stem cells from a tissue of origin, such as bone marrow. For example, a releasing agent increases the number of bone marrow-derived stem cells and/or hematopoietic stem cells in the peripheral blood. In another embodiment, the releasing agent affects the number of stem cells, such as CD34^{high} (CD34+) cells, circulating in the peripheral blood.

“Stem cells” as used herein are cells that are not terminally differentiated and are therefore able to produce cells of other types. Characteristic of stem cells is the potential to develop into mature cells that have particular shapes and specialized functions, such as heart cells, skin cells, or nerve cells. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells”, as used herein, refers to pluripotent stem cells and multipotent stem cells capable of self-renewal and differentiation. “Bone marrow-derived stem cells” are the most primitive stem cells found in the bone marrow which can reconstitute the hematopoietic system, possess endothelial, mesenchymal, and pluripotent capabilities. Stem cells may reside in the bone marrow, either as an adherent stromal cell type, or as a more differentiated cell that expresses CD34, either on the cell surface or in a manner where the cell is negative for cell surface CD34. “Adult stem cells” are a population of stem cells found in adult organisms with some potential for self-renewal and are capable of differentiation into multiple cell types. Other examples of stem cells are marrow stromal cells (MSCs), HSC, multipotent adult progenitor cells (MAPCs), very small embryonic-like stem cells (VSEL), epiblast-like stem cell (ELSC) or blastomere-like stem cell (BLSC).

“Stem cell circulation agent” (SCCA), “mobilization agent”, and/or “mobilization factor” as used herein refers to one or more compounds, antibodies, nucleic acid molecules, proteins, polysaccharides, cells, or other molecules, including, but not limited to, neuropeptides and other signaling molecules, that affects the release, circulation, homing and/or migration of stem cells from the circulatory system into tissue or organ. A molecular agent may be a naturally occurring molecule or a synthetic molecule. Examples of mobilization agents include “releasing agents”, wherein a releasing agent is capable of promoting the egress of stem cells from a tissue of origin and also “migration agents”, wherein a migration agent is capable of promoting the process of a cell moving from the circulatory system into a tissue or organ.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Succulent” as used herein refers to all species of plants within the family Agavaceae, Cactaceae, Crassulaceae, Aizoaceae, Apocynaceae, Didiereaceae, Euphorbiaceae, Asphodelaceae, Portulacaceae. This further includes plants known to possess storage organs adapted for water retention, wherein the storage organs are located in the leaf, stems, roots, or any other location.

“Therapeutically effective amount” as used herein refers to the quantity of a specified composition, or active agent in the composition, sufficient to achieve a desired effect in a subject being treated. For example, this can be the amount effective for enhancing migration of stem cells that replenish, repair, or rejuvenate tissue. In another embodiment, a “therapeutically effective amount” is an amount effective for enhancing trafficking of stem cells, such as increasing release of stem cells, as can be demonstrated by elevated levels of circulating stem cells in the bloodstream. In still another embodiment, the “therapeutically effective amount” is an amount effective for enhancing homing and migration of stem cells from the circulatory system to various tissues or organs, as can be demonstrated be decreased level of circulating stem cells in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Trafficking” as used herein refers to the process of movement of a cell from the tissue of origin, traveling within the circulatory or immune system, and localization towards a site within a tissue and/or organ. Trafficking also includes stem cell mobilization, beginning with release from a tissue of origin, such as egress of stem cells from bone marrow. Trafficking further includes movement of a cell from the tissue of origin, homing by adhesion to the endothelium, transmigration, and final migration within the target tissue and/or organ. Furthermore, trafficking may include the process of movement of a cell of the immune system. One specific, non-limiting example of trafficking is the movement of a stem cell to a target organ, also referred to as migration. Another specific, non-limiting example of trafficking is the movement of a B-cell or a pre-B-cell leaving the bone marrow and moving to a target organ.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

As described, stem cells are unique cells that possess the capacity to differentiate into more specialized cells. One particular type of stem cell, hematopoietic stem cells (HSCs), are capable of differentiating into many different types of blood cells. In addition, HSCs typically reside in the bone marrow, where proliferation and self-renewal of the cells allows HSCs to be involved in the support and maintenance of the hematopoietic system. Existing scientific literature has chiefly focused on HSCs' potential to develop into hematopoietic lineage cells derivatives. Emerging evidence has further identified the capacity for HSCs to also differentiate into non-hematopoietic, tissue specific cells. Recently, HSCs have been found to possess the capacity to differentiate into a variety of tissue-specific cell types, such as myocytes, hepatocytes, osteocytes, glial cells, and neurons. As a result, HSCs form blood and immune cells that are responsible for constant maintenance and immune protection of virtually every cell type of the body.

Similarly, bone marrow stem cells (BMSCs) were recently shown to have significant capability to become cells of other tissues. In the bone marrow, stem cells duplicate using a process known as “asymmetrical cellular division” according to which the two daughter cells are not identical; one cell retains the original DNA and remains in the bone marrow whereas the other cell contains the DNA copies and is released in the blood where it migrates into various tissues in need of repair. BMSCs have been traditionally considered to have little potential for plasticity, being limited in their development to red blood cells, lymphocytes, platelets, bone and connective tissue. However, much scientific work has been published over the past few years that demonstrates the exceptional plasticity of BMSC. For example, after transplantation, BMSCs and HSCs were shown to have the ability to become muscle cells, heart cells, endothelium capillary cells, liver cells, as well as lung, gut, skin, and brain cells. As a further illustrative example, some studies report the ability of HSC to become liver cells upon contact with specific liver-derived molecules, but this process took place within hours. Briefly, HSCs were co-cultured with either normal or damaged liver tissue separated by a semi-permeable membrane (pores large enough to let molecules pass through, but small enough to prevent the passage of cells from one compartment to the other, pore size 0.4 μm). Using immunofluorescence assay methods to detect molecules specific for either HSCs (CD45) or liver cells (albumin), the researchers could follow the transformation of the population of cells placed in the upper compartment. When HSCs were cultured alone for 8 hours, they only expressed CD45 and no albumin, indicating that no HSCs had differentiated into liver cells. However, when HSCs were exposed to injured liver tissue, they rapidly became positive for albumin. Over time, the population of cells positive for CD45 began to decrease as the population positive for albumin began to increase. Albumin-positive cells were seen as early as 8 hours into the procedure and increased in frequency to 3.0% at 48 hours. The conversion was minimal and delayed when HSCs were exposed to undamaged liver (control for injury).

Because HSCs and BMSCs play an important role in the healing and regenerative processes of various tissues and organs in the body beyond their traditional role in maintaining hematopoietic and immune systems of the body, activation and enhancement of stem cell trafficking may amplify these physiological processes and provide a potential therapy for various pathologies. The classic source of HSCs and BMSCs is bone marrow, which includes hip, ribs, sternum and other bone structures. Bone provides a unique regulatory microenvironment for HSCs and BMSCs, including interaction with a specific mesenchymal cell type (the osteoblast), extracellular matrix glycoproteins and a uniquely rich mineral signature. This stem cell “niche” contains a great deal of critical molecular interactions which guide the response of stem cells to specific physiological conditions. The niche may be an important focal point for changes in the state of tissue that result in a change in the regenerative processes rooted in stem cell activity. (Adams and Scadden, 2006)

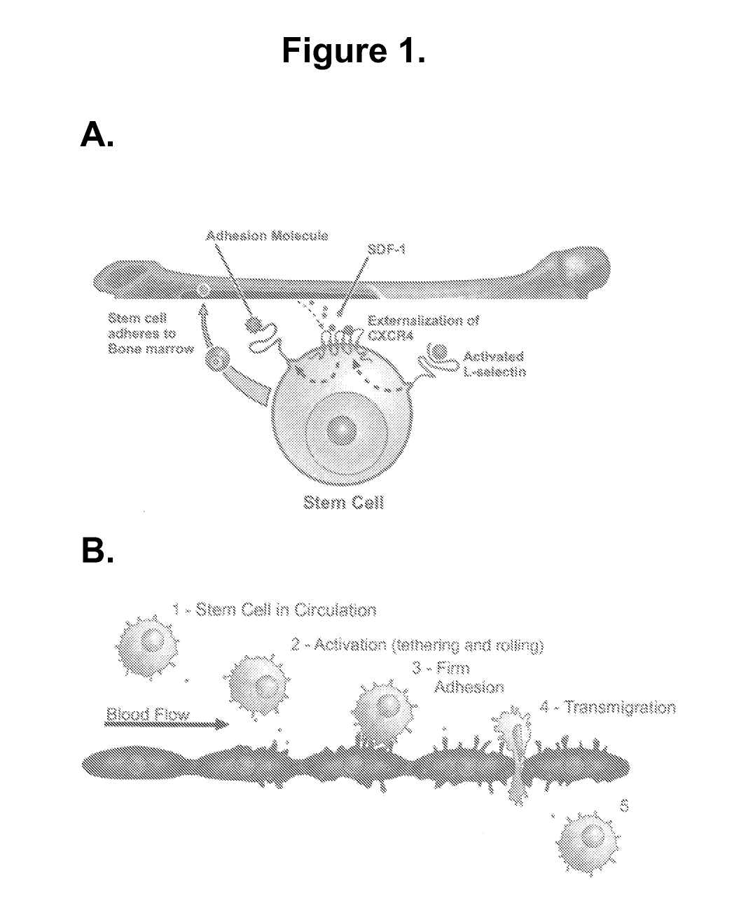

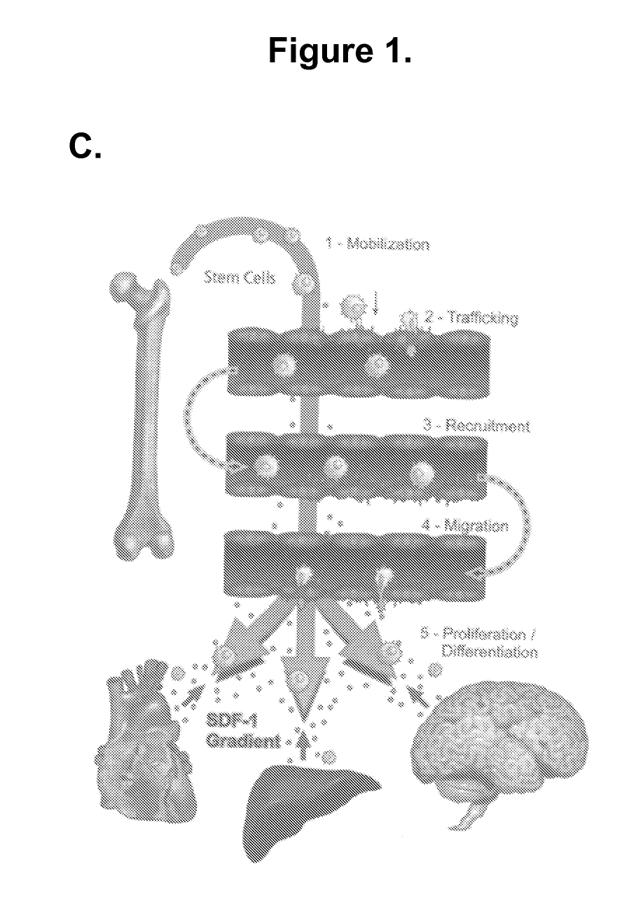

Beyond populations of HSCs found in bone marrow, HSCs are also present in the peripheral bloodstream of normal, healthy persons. It has been known for decades that a small number of stem and progenitor cells circulate in the bloodstream, but more recent studies have shown that greater numbers of HSCs can be coaxed into mobilization from marrow to blood by injecting the donor with a cytokine, such as granulocyte-colony stimulating factor (G-CSF). Despite this advance, the natural process by which stem cells are released from bone marrow and migrate towards a site within tissue and/or an organ is not fully understood. A leading model involves the chemokine, Stromal-Derived Factor-1 (SDF-1) and its specific receptor, CXCR4. In this capacity, the binding of SDF-1 to CXCR4, leads to adherence of stem cells to bone marrow through increased expression of adhesion molecules on the cell membrane surface. Disruption of adhesion of stem cells to bone marrow thus promotes mobilization of stem cells into the peripheral bloodstream. (FIG. 1C) Some factors such as G-CSF or IL-8 may interfere with adhesion through elevated activation of protelytic enzymes or degradation of the SDF-1 ligand. Other types of molecules, such as L-selectin blockers, may instead down-regulate CXCR4 expression which in turn reduces stem cell adhesion to the bone marrow environment. Generally speaking enhancing binding of SDF-1 to CXCR4 promote adherence, therefore L-selectin blockers such as sulfated fucans, which reduces CXCR4 expression, can trigger stem cell mobilization.

Stem cells circulating in the peripheral bloodstream are recruited to sites of tissue in need of repair and regeneration through homing and extravasation. This mobilization of stem cells into the bloodstream and subsequent migration to the site of tissue injury results from a combination of mechanical and chemoattractant signals. Mechanical force or other factors may activate L-selectins on the surface of stem cells. Activation of L-selectins, in turn, may promote elevated expression of the receptor, CXCR4. Cells at the site of tissue injury may also secrete SDF-1 ligand, thereby attracting stem cells expressing receptor CXCR4 to the injury site. The interaction of SDF-1 and CXCR4 promotes sufficient adhesion to halt circulation of a stem cell in the peripheral blood stream. (FIG. 1B) Based on this model, L-selectin blockers such as sulfated fucans, may possess a critical capacity to mobilize HSCs into the bloodstream, with subsequent homing, extravasation and migration into tissue promoting regenerative maintenance and repair of cells and tissues in an organism. Whereas G-CSF is released from injured tissue and its presence in the bloodstream triggers HSC release from bone marrow, dietary supplements composed of L-selectin blockers may possibly support the phenomenon of natural regeneration and repair in the body.

Aloe.

One example of plant species from which plant stem cells can be isolated and cultured as cell lines includes the plants from the Aloe genus. Members of the Aloe genus have been used in cosmetic and medicinal applications, and certain plants, such as Aloe vera have been dubbed the ‘Lily of the desert’, ‘Plant of immortality’, and ‘The medicine plant’. Components extracted from Aloe have been used in healing/wound repair, anti-inflammatory and antioxidant effects, among many other applications. These effects may result from the biological components such as glucomannan and acemannan present in plants from the Aloe genus. These biologically active components in Aloe are often found in the clear, thick gel found in the inner portions of leaves from Aloe plants. While this gel is 99 percent water, it is known to contain a wide range glycoproteins and polysaccharides, such as Among known biologically active components are glucomannan (moisturizer), acemannan (modulation of immune function, including macrophage activation and cytokine production), bradkininase (anti-inflammatory), magnesium lactate (antipruritic effects). However, the application of Aloe extracts with a view towards use in promoting stem cell regeneration and repair is largely unknown.

Of particular interest is the Aloe macroclada species endemic to Madagascar, unlike better-known species such as Aloe ferox in southern Africa and Aloe vera in northern Africa. The stemless Aloe macroclada is widespread in grasslands in southern central Madagascar to 1500 m, with broad leaves, often tinged red and an upright, unbranched inflorescence, and grows in highlands (altitude above 1,000 m) with specific climate conditions: average temperature (14-22° C.) and rainfall (more than 1,000 mm) with six months of dry season. Whereas Aloe gel has been used for the described medicinal purposes, whole Aloe macroclada plants or structures can be compressed into crude preparations or capsules, suitable for ingestion as exists among certain traditional medicine practices in Madagascar, wherein the indigenous use of these compositions is through pellets, sometimes containing burned plant material of Aloe macroclada. The mechanisms for such therapeutic approaches are not understood, but suggest potential for biologically active components to exist in not only in Aloe gel, but in roots, leaf, or other plant Aloe structures. In some embodiments, it may be of interest to extract, isolate or purify such components to enhance their therapeutic effects.

Aloe Extracts.

Extracts may be prepared according to any number of methods known to one of ordinary skill in the art. Generally, extraction procedures involves contacting solid plant material with a solvent with adequate mixing and for an amount of time to ensure adequate exposure of the solid materials to the solvent to enable extract components to be taken up by the solvent. Solvents may be aqueous, alcoholic, and organic solvents for use in extracting components of varying polar and non-polar character. As an example, plant material can be crushed mechanically and placed in contact with aqueous TRIS-HCl buffer at pH 6-8, from 0.5-8 hours, at a temperature between 4-50° C. to extract aqueous components from the plant material. Following contact of the solid plant materials with the liquid solvent, solid insoluble matter is separated, generating a liquid as a crude extract preparation and a solid fraction. Separation of the liquid and solid fractions may be performed according to a variety of methods including centrifugation, filtration, chromatography, or any other methods known to one of skill in the art. Following separation of a liquid fraction, such as decanting of an aqueous solvent following centrifugation, the remaining solid can be contacted with a second solvent, such as an alcoholic solvent and cosolvent, such as methanol or water. Centrifugation again provides a means of separate insoluble solid plant material and soluble components in the liquid fraction. These components in the alcoholic extract may be recovered using a lypophilizer, speed vac, rotary evaporator or a vacuum pump, and dried. Organic extracts may further be obtained by shaking the residual solid, in the presence of an of a suitable organic solvent, such as dimethylsulfoxide or dichloromethane. Lipid fractions may also be obtained by addition of highly lipophilic agents, such as addition of liposomes, to extract nonpolar biologically active components. In each case, these various separation processes can be used to isolate a biologically active component of interest. For example, biologically active components known to be present in Aloe include glucomannan, acemannan, bradykinase, magnesium lactate, salicylic acid, antiprostaglandins, maloyl glucans, veracylglucan A, veracylglucan B, veracylglucan C, mannose-6-phosphate, di(2-ethylhexyl) phtalate (DEHP), calcium isocitrate, aloin, aloe-emodin and other anthraquinone glycosides.

Polygonum multiflorum.

The dried root tuber of Polygonum multiflorum plant, also known as fleeceflower root, has been used as a traditional Chinese medicine called He shou wu, this medication gaining notoriety in TCM from a tale of a famous Chinese military officer condemned to death and jailed without food or drink. Surviving by consuming the leaves and roots of the vinelike weed, Polygonum multiflorum, the officer's captors later found his remains as still having lustrous black hair. While the origins of this tale are apocryphal, they serve to illustrate the long-held notion that Polygonum multiflorum possesses important properties for tapping into the regenerative and restorative potential of the body. Recent scientific studies have confirmed that extracts of Polygonum multiflorum are indeed capable of promoting hair follicle growth, through increased expression of sonic hedgehog (Shh) and β-catenin expression—two important pathways involved in both early embryogenesis and maintaining stem cell identity.

Further analysis of Polygonum multiflorum extracts have confirmed this plant to be a rich source of bioactive compounds, two notable examples being anthraquinones and derivatives and hydroxyy stilbenes. Anthraquinones and derivatives have served as the basis for antimalarial, laxative, and chemotherapy treatments. Hydroxyl stilbenes, such as 2,3,5,4′-tetrahydroxystilbene-2-O-β-D-glucoside, have been show to provide important neuroprotective effects warding off symptoms of different neurodegenerative diseases. Together, these results indicate that components of Polygonum multiflorum extracts possess important properties for healing and regenerating the body, possibly by modulating inflammation, reducing risk of cancer proliferation, and/or providing protective effects for cells, tissues, and organs of the body.

While effects of these components in Polygonum multiflorum is somewhat understood for certain specific conditions, there is much less knowledge about how components of Polygonum multiflorum may specifically influence stem cell activity in the body. This is surprising given that, as described, stem cells play an integral role in the body's natural healing and regeneration mechanisms. One of the few existing studies on the subject indicates that Polygonum multiflorum extracts promotes proliferation of stem cells and progenitors, as shown by an increase in the number of bone marrow stem cells and lymphoid progenitors following administration of Polygonum multiflorum extracts in mice. Similarly, U.S. patent application Ser. No. 12/006,221 describes an increase in GM-CSF and stem cell factor (SCF) expression following administration in mice. These results present intriguing questions about potential effects of Polygonum multiflorum extracts on stem cell activity, given that both GM-CSF and SCF are implicated as playing important roles in stem cell migration and mobilization, as described above.

Fucoidan.

Fucoidan is a sulfated fucan polysaccharide L-selectin agonist that was documented to promote the egress of HSCs from compartments in bone marrow into the peripheral blood stream upon intravenous injection, although this effect seemed unrelated to its stimulation of L-selectin. Circulation of HSCs in the peripheral bloodstream is a critical step in promoting the stem cell regeneration and repair mechanisms in the body. As a sulfated fucan, fucoidan is found in various species of algae. Other sulfated fucans have also been found in animal species, such as echinoderms (e.g., sea urchins and sea cucumbers).

Despite in vivo data in animal models that demonstrate significantly elevated levels of HSCs following intravenous fucoidan administration, observations of positive clinical effects in human subjects are much more limited. Reported studies have shown that the percentage of HSCs expressing an important trafficking receptor, CXCR4, increased significantly following 4 days of oral fucoidan administration. However, only a slight change was observed in the absolute number of HSCs circulating in peripheral blood.

As described, fucoidan (also known as fucoidin or fucansulfate in the art) is a sulfated fucose polysaccharide L-selectin ligand. Selectin activity depends on important carbohydrate or polypeptide modifications such as sialylation, fucosylation, and sulfation. The presence of binding sites for sulfated fucans such as fucoidan on P- and L-Selectin has been demonstrated to be at least partially the mechanism by which fucoidan promotes detachment of HSCs from BM. Perhaps more significantly, sulfated fucans such as fucoidan, have been shown to displace SDF-1 sequestered on endothelial surfaces or bone marrow through completive binding to a heparin-binding domain present on SDF-1. Occupation of the heparin-binding site of SDF-1 by fucoidan prevents tethering to cell surfaces, thereby increasing circulating SDF-1 levels in plasma. Without being bound by any particular theory, the enhanced levels of SDF-1 ligand in the bloodstream may thus promote egress of CXCR4 receptor expressing HSCs from the BM. (FIG. 1C) Based on this model, the inventors hypothesized that L-selectin ligand, such as fucoidan, may possess a critical capacity to mobilize HSCs and oral administration of dietary supplements composed of fucoidan may best support natural regeneration and repair in the body.

Compelling in vivo data in animal models demonstrates significantly elevated levels of circulating HSCs following intravenous (IV) fucoidan administration in mice and primates, although significant drawbacks would present limitations for human therapeutic use. Recent reports have shown a dramatic 12-fold increase in levels of circulating HSCs, HSC progenitors and derivative cell types (including erythroid burst forming units, granulocyte, erythroid, macrophage, megakaryocyte colony forming units, granulocyte, erythroid, macrophage, and granulocyte macrophage colony-forming units) compared to untreated controls, 3 hours following injection of fucoidan (source unknown) into mice. Similar results of sustained elevation in levels of HSCs, HSCs progenitors and derivative cell types, were reported after daily injections for 3 days. Injection of fucoidan in primates has also been demonstrated to increase HSCs and HSC-derivative levels by 11-26 fold after 6 hours after administration, with sustained elevation still observable up to 24 hours later. Despite these positive observations, several challenges could impede therapeutic use of fucoidan in human subjects. The temporary and transitory effect of elevated HSCs circulating and bone marrow-derived stem cells may fail to fully realize the positive clinical benefits of stem cell regenerative and repair mechanisms, since sustained or repeated periods of elevation may be needed to enable stem cell homing and extravasation processes that underlie therapeutic stem cell activity. This limitation is further compounded in view of the difficulty and inconvenience of routinely administering IV injections.

Existing observations in human subjects are limited and available data on oral fucoidan administration in humans does not mirror the positive clinical effects of animal studies using IV injection. Reported studies by others have shown that the percentage of HSCs expressing an important trafficking receptor, CXCR4, increased significantly (45% to 90%) after 12 days of oral fucoidan administration (3 grams daily of 10% w/w or 75% w/w fucoidan extracts from Undaria pinnatifida). However, only a slight change (˜12%) was observed in the absolute number of HSCs circulating in peripheral blood (maximal effect was 1.64 to 1.85 cells/μl after 4 days of fucoidan extract administration). Importantly, for therapeutic applications involving oral administration, fucoidan is capable of surviving acidic conditions in the stomach and does not demonstrate adverse side effects. This is consistent with reports that catalytic fucoidinase, which metabolizes fucoidan, is found only in marine interverbrates and not terrestrial mammals. This may provide an vital therapeutic benefit of high persistence and stability of an administered sulfated fucan, including fucoidan, for sustained therapeutic effect. It is particularly ideal for oral uses where diffusion into the bloodstream must first survive enzymatic processing in the mouth, esophagus, and intestines, in addition to the highly acidic conditions of the stomach.

The inventors have discovered that the source of fucoidan and appropriate dosing regimens are critical features for promoting HSC mobilization through oral fucoidan administration. Fucoidan is a member of the broader class of sulfated fucans, which are polysaccharides rich in L-fucose and obtained primarily from two sources: algae and marine invertebrates. Sulfated fucans obtained from these two sources differ greatly in composition and structure. This diversity of molecular structure further exists across fucoidans from different species of algae. While generally described as ˜20,000 molecular weight polysaccharide composed of L-fucose, exact fucoidan structures depend in-part, on the source organism. As example, the most well-studied fucoidan from F. vesculosus, is reported to be composed primarily of L-fucose with α(1→3) glycosidic bonds and sulfate groups at position 4, with sulfated fucose branches every 5 units. In contrast, fucoidan from a different algae, Ascophylum nodosum, has a large proportion of repeating α(1→3) and α(1→4) glycosidic bonds that alternate for oligosaccharide formation, possibly with few sulfated branching points as showing in nuclear magnetic resonance (NMR) studies (Berteau, 2003). In sum, fucoidans from different species are structurally distinct, heterogeneous and diverse.

The present invention provides new compositions and methods for providing a wide range of clinical and physiological benefits to a subject in need thereof by the administration of a mobilization agent. While not wishing to be bound by any particular theory, the inventors believe that the beneficial and other physiological results obtained through administration of the inventive compositions result from enhancing stem cell trafficking and migration that follows the administration of the mobilization agent.

Described herein are compositions including a mobilization agent with one or more components selected from the group including: Aloe or extracts thereof, Polygonum multiflorum or extracts thereof, Lycium barbarum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items. The mobilization agents may be combined together in one or more compositions or they may be administered or consumed separately as part of a regimen. They may have individual physiological effects, additive effects and/or synergistic effects with one another, such as serving as both a releasing agent and migration agent. In some embodiments, the mobilization agent is capable of functioning as a migration agent, promoting the process of a cell moving from the circulatory system into a tissue or organ. In some embodiments, the mobilization agent is capable of functioning as a releasing agent, promoting the release and egress of stem cells from a tissue of origin. In various embodiments, the composition is a pharmaceutical composition including the above components and a pharmaceutically acceptable carrier.

In one embodiment, a mobilization agent is administered to a subject, for example Aloe, though the subject may be provided a mixture of Aloe and other mobilization agents. In some embodiments, the subject consumes and digests whole Aloe plant. The plant may be fresh, frozen, freeze-dried, dehydrated, or preserved in some other manner. Therefore, Aloe, as described herein, encompasses both whole plant and extracts thereof. In one embodiment, the mobilization agent is an extract of Aloe, or an isolated component or compound extracted from Aloe, such as a compound found in a polysaccharide-rich fraction of Aloe extract. Aloe can be provided alone as an isolated or purified substance, or may be part of a composition including a pharmaceutically acceptable carrier. In one embodiment, Aloe is capable of functioning as a migration agent. In certain embodiments, Aloe is Aloe macroclada.

In one embodiment, a mobilization agent is administered to a subject, for example Polygonum multiflorum, though the subject may be provided a mixture of Polygonum multiflorum and other mobilization agents. In some embodiments, the subject consumes and digests whole Polygonum multiflorum. The plant may be fresh, frozen, freeze-dried, dehydrated, or preserved in some other manner. Therefore, Polygonum multiflorum, as described herein, encompasses both whole berry and extracts thereof. In one embodiment, the mobilization agent is an extract of Polygonum multiflorum, or an isolated component or compound extracted from Polygonum multiflorum, such as a compound found in a polysaccharide-rich fraction of Polygonum multiflorum extract. Polygonum multiflorum can be provided alone as an isolated or purified substance, or may be part of a composition including a pharmaceutically acceptable carrier.

In alternative embodiments, an extract of the algae is provided or administered to the subject. In another embodiment, the algae encompasses both whole plant and extracts thereof. In another embodiment, the algae can be provided alone as an isolated or purified substance, or may be part of a composition including a pharmaceutically acceptable carrier. In another embodiment, the extract is a highly sulfated, polyanionic soluble fiber. In one embodiment, the extract is an isolated fucoidan. In a different embodiment, the fucoidan is purified following isolation. In an alternative embodiment, a polysaccharide fraction is administered to the subject. In another embodiment, the highly sulfated, polyanionic soluble fiber is administered to the subject. In one, the isolated fucoidan is administered to the subject. In a different embodiment, the purified fucoidan is administered to the subject. In one embodiment, Undaria pinnatifida is capable of functioning as a releasing agent after administration to a subject.

In one embodiment, a mobilization agent is administered to a subject, for example Lycium barbarum, though the subject may be provided a mixture of Lycium barbarum and other mobilization agents. In some embodiments, the subject consumes and digests whole Lycium barbarum berries. The berries may be fresh, frozen, freeze-dried, dehydrated, or preserved in some other manner. Therefore, Lycium barbarum, as described herein, encompasses both whole berry and extracts thereof. In one embodiment, the mobilization agent is an extract of Lycium barbarum, or an isolated component or compound extracted from Lycium barbarum, such as a compound found in a polysaccharide-rich fraction of Lycium barbarum extract. Lycium barbarum can be provided alone as an isolated or purified substance, or may be part of a composition including a pharmaceutically acceptable carrier.

In one embodiment, colostrum is administered to a subject, though the subject may be provided a mixture of colostrum and other mobilization agents. In some embodiments, the subject consumes and digests whole colostrum. The colostrum may be fresh, frozen, freeze-dried, dehydrated, or preserved in some other manner. Therefore, colostrum, as described herein, encompasses both whole colostrum and extracts thereof. In one embodiment, the mobilization agent is an extract of colostrum, or an isolated component or compound extracted from colostrum, such as a compound found in a protein-rich fraction of colostrum extract colostrum can be provided alone as an isolated or purified substance, or may be part of a composition including a pharmaceutically acceptable carrier.

In one embodiment, mushroom or a blend of mushrooms is administered to a subject, though the subject may be provided a mixture of mushrooms and other mobilization agents. In some embodiments, the subject consumes and digests whole mushrooms. The mushrooms may be fresh, frozen, freeze-dried, dehydrated, or preserved in some other manner. Therefore, mushrooms, as described herein, encompass both whole mushrooms and extracts thereof. In one embodiment, the agent is Cordyceps sinensis or an extract thereof. In one embodiment, the mobilization agent is Ganoderma lucidum or an extract thereof. In one embodiment, the mobilization agent is Hericium erinaceus or an extract thereof. Mushrooms can be provided alone as isolated or purified substances, or may be part of a composition including a pharmaceutically acceptable carrier.

In one embodiment, algae is administered to a subject, though the subject may be provided a mixture of algae and other mobilization agents. In some embodiments, the subject consumes and digests whole algae. The algae may be fresh, frozen, freeze-dried, dehydrated, or preserved in some other manner. Therefore, algae, as described herein, encompass both whole algae and extracts thereof. In one embodiment, the mobilization agent is Chordaria cladosiphon or an extract thereof. Algae can be provided alone as isolated or purified substances, or may be part of a composition including a pharmaceutically acceptable carrier. In one embodiment, algae, Chordaria cladosiphon is capable of functioning as a migration agent.

In one embodiment, spirulina is administered to a subject, though the subject may be provided a mixture of spirulina and other mobilization agents. In some embodiments, the subject consumes and digests whole spirulina. The spirulina may be fresh, frozen, freeze-dried, dehydrated, or preserved in some other manner. Therefore, spirulina, as described herein, encompasses both whole spirulina and extracts thereof. In one embodiment, the mobilization agent is Arthrospira platensis, Arthrospira maxima, or an extract thereof. Spirulina can be provided alone as an isolated or purified substance, or may be part of a composition including a pharmaceutically acceptable carrier.

In various embodiments, the dosage of the each of the one or more mobilization agents in the composition can include 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. For example, a representative set of dosages in the composition are shown in Table 1. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Aloe or extracts thereof, Polygonum multiflorum or extracts thereof, Lycium barbarum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items. In certain embodiments, Aloe is Aloe macroclada. In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In various embodiments, the total dosage amount is administered daily for one or more days, or multiple times in a single day.

The present invention further provides a method of enhancing the trafficking of stem cells in a subject. In one embodiment, the level of trafficking of stem cells relates to the number of circulating hematopoietic stem cells (HSCs) in the peripheral blood of a subject. In another embodiment, the level of trafficking of stem cells relates to the number of circulating bone marrow-derived stem cells in the peripheral blood of a subject. In various embodiments, enhancing the trafficking of stem cells in a subject, includesadministering a therapeutically effective amount of a mobilization agent, thereby increasing the release, circulation, homing and/or migration of stem cells in the subject, regardless of the route of administration.

In another embodiment, the method provided herein enhances the trafficking of stem cells in a subject, including administering a therapeutically effective amount of a composition containing one or more of the following components selected from the group including: Aloe or extracts thereof, Polygonum multiflorum or extracts thereof, Lycium barbarum or extracts thereof, colostrum or extracts thereof, spirulina or extracts thereof, Arthrospira platensis or extracts thereof, Arthrospira maxima or extracts thereof, fucoidan or extracts thereof, Chordaria cladosiphon or extracts thereof, Hericium erinaceus or extracts thereof, Ganoderma lucidum or extracts thereof, and/or Cordyceps sinensis or extracts thereof, thereby enhancing the trafficking of stem cells in the subject. In one embodiment, enhancement of stem cell trafficking may be measured by assaying the response of stem cells to a particular dose of a composition containing one or more of the following components selected from the group including: Aloe or extracts thereof, Lycium barbarum or extracts thereof, colostrum or extracts thereof, spirulina or extracts thereof, Arthrospira platensis or extracts thereof, Arthrospira maxima or extracts thereof, fucoidan or extracts thereof, Chordaria cladosiphon or extracts thereof, Hericium erinaceus or extracts thereof, Ganoderma lucidum or extracts thereof, and/or Cordyceps sinensis or extracts thereof, thereby enhancing the trafficking of stem cells in the subject.

In another embodiment, a method of enhancing the trafficking of stem cells in a subject includes a transient increase in the population of circulating stem cells, such as stem cells following administration of a mobilization agent. In one embodiment, the stem cells are hematopoietic stem cells (HSGs). In another embodiment, the stem cells are bone marrow-derived stem cells. In various embodiments, the stem cells are CD45^{dim} CD34⁺, CD34⁺, CD34⁺ KDR⁻, or CD45-CD31+KDR+, CD34+CD133-, CD34+CD133+, or express various sub-combinations of these markers. In another embodiment, the administration of an extract of a mobilization agent leads to an increase in CXCR4 expression on circulating stem cells. In one embodiment, providing a mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration. In various embodiments, release of stem cells into the circulation from about 1, 2, or 3 hours following administration. In another embodiment, released stem cells enter the circulatory system and increase the number of circulating stem cells within the subject's body. In another embodiment, the percentage increase in the number of circulating stem cells compared to a normal baseline may about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

In another embodiment, a method of enhancing the trafficking of stem cells in a subject includes a transient decrease in the number of circulating stem cells within the subject's body. In another embodiment, a method of enhancing the trafficking of stem cells in a subject includes inducing a transient decrease in the population of circulating stem cells, such as stem cells. In one embodiment, the stem cells are hematopoietic stem cells (HSGs). In another embodiment, the stem cells are bone marrow-derived stem cells. In various embodiments, the stem cells are CD45^{dim} CD34⁺, CD34⁺, CD34⁺ KDR⁻, or CD45-CD31+KDR+, CD34+CD133-, CD34+CD133+, or express various sub-combinations of these markers. In another embodiment, the administration of an extract of a mobilization agent leads to an increase in CXCR4 expression on circulating stem cells. In one embodiment, providing a mobilization agent to a subject will enhance migration of that subject's stem cells within a certain time period, such as less than about 5 hours, less than about 4 hours, less than about 2 hours, or less than about 1 hour following administration. In other embodiments, the mobilization agent is colostrum, mushroom polysaccharides including Cordyceps sinensis, Hericium erinaceus, Ganoderma lucidum, fucoidan including Chordaria cladosiphon, spirulina, including Arthrospira platensis, and/or Arthrospira maxima. In various embodiments, the percentage decrease in the number of circulating stem cells compared to a normal baseline may about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier.

In one embodiment, administration of a mobilization agent results in the migration of stem cells from the circulation to tissues from about 1 to about 3 hours following administration. Circulating stem cells will leave the circulatory system, thus decreasing the number of circulating stem cells within the subject's body. The percentage decrease in the number of circulating stem cells compared to a normal baseline may be about 15%, about 30%, about 50% or greater than about 75% decrease as compared to a control. In one embodiment, the control is a base line value from the same subject. In another embodiment, the control is the number of circulating stem cells in an untreated subject, or in a subject treated with a placebo or a pharmacological carrier.

In another embodiment, administration a mobilization agent increases the rate of homing of stem cells measured by a transient decrease in the number of circulating stem cells within the subject's body. The percentage decrease in the number of circulating stem cells compared to a normal baseline may be about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier. In another embodiment, the administration of an extract of a mobilization agent leads to an increase in CXCR4 expression on circulating stem cells.

In various embodiments, administering a therapeutically effective amount of a composition includes oral administration of a dosage containing one or more mobilization agents in the amount of 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. For example, a representative set of dosages in the composition are shown in Table 1. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Aloe or extracts thereof, Polygonum multiflorum or extracts thereof, Lycium barbarum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items. In certain embodiments, Aloe is Aloe macroclada. In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In various embodiments, the total dosage amount is administered daily for one or more days, or multiple times in a single day.

In some embodiments, the subject administered a mobilization agent is healthy. In other embodiments, the subject is suffering from a disease or physiological condition, such as immunosuppression, chronic illness, traumatic injury, degenerative disease, infection, or combinations thereof. In certain embodiments, the subject may suffer from a disease or condition of the skin, digestive system, nervous system, lymph system, cardiovascular system, endocrine system, or combinations thereof. In specific embodiments, the subject suffers from osteoporosis, Alzheimer's disease, cardiac infarction, Parkinson's disease, traumatic brain injury, multiple sclerosis, cirrhosis of the liver, any of the diseases and conditions described in the Examples below, or combinations thereof. Administration of a therapeutically effective amount of a mobilization agent may prevent, treat and/or lessen the severity of or otherwise provide a beneficial clinical benefit with respect to any of the aforementioned conditions, although the application of the inventive methods and use of the inventive mobilization agent is not limited to these uses. In various embodiments, the novel compositions and methods find therapeutic utility in the treatment of, among other things, skeletal tissues such as bone, cartilage, tendon and ligament, as well as degenerative diseases, such as Parkinson's and diabetes. Enhancing the release, circulation, homing and/or migration of stem cells from the blood to the tissues may lead to more efficient delivery of stem cells to a defect site for increased repair efficiency. The novel compositions and methods of the present invention may also be used in connection with gene therapeutic approaches.

The present invention further provides various compositions for administration to a subject. In one embodiment, the administration is topical, including ophthalmic, vaginal, rectal, intranasal, epidermal, and transdermal. In one embodiment, the administration is oral. In one embodiment, the composition for oral administration includes powders, granules, suspensions or solutions in water or non-aqueous media, capsule, sachets, tablets, lozenges, or effervescents. In another embodiment, the composition for oral administration further includes thickeners, flavoring agents, diluents, emulsifiers, dispersing aids or binding agents.

Described herein are mobilization agents and methods of using mobilization agents towards promoting stem cell trafficking. Further described herein are migration agents and method of using migration agents to promote the process of stem cells moving from the circulatory system into a tissue or organ. Also described herein are releasing agents and methods of using releasing agents to promote egress of stem cells from a tissue of origin. Also described herein is a method of oral administration of mobilization agents which result in a significant release of HSCs into peripheral blood circulation. The inventors have demonstrated effective administration of stem cell mobilization agents, thereby achieving a safe, convenient and effective method to enhance stem cell-related maintenance and repair in the human body. Although the pathology of stem cells is of great importance and interest, and pertains to the subject matter disclosed herein, the underlying scope of this invention is that the release, circulation, homing and/or migration of stem cells from the blood to tissues is of significance in repairing injured tissue and maintaining the vitality and health of existing tissue. Thus, the importance of developing methods and compositions for achieving this end are among the foci and aims of the present invention.

Accordingly, the present invention provides novel compositions and methods for, among other things, enhancing natural tissue healing and renewal in the body by supporting the trafficking of stem cells. Furthermore, the present invention provides novel compositions and methods for preventing, slowing or otherwise diminishing the development of health problems in a mammal by promoting trafficking of stem cells in the mammal. The compositions and methods disclosed herein may further increase regeneration of existing tissue by supporting the release, circulation, homing and/or migration of stem cells into tissue, therefore supporting the process of tissue repair.

### EXAMPLES

The following examples are provided to better illustrate the claimed invention and are not to be interpreted as limiting the scope of the subject matter. To the extent that specific materials are mentioned, it is merely for purposes of illustration and is not intended to limit the invention. One skilled in the art may develop equivalent means, compositions or reactants without the exercise of inventive capacity and without departing from the scope of the present invention.

### Example 1

### Production and Preparation of L. barbarum

Polysaccharides from Lycium barbarum were prepared by the method of Luo et al. (2004). The dried fruit samples (100 g) were ground to fine powder and put in 1.5 l of boiling water and decocted for 2 h by a traditional method for Chinese medicinal herbs. The decoction was left to cool at room temperature, filtered and then freeze-dried to obtain crude polysaccharides.

The dried crude polysaccharides were refluxed three times to remove lipids with 150 ml of chloroform:methanol solvent (2:1) (v/v). After filtering the residues were air-dried. The result product was extracted three times in 300 ml of hot water (90° C.) and then filtered. The combined filtrate was precipitated using 150 ml of 95% ethanol, 100% ethanol and acetone, respectively. After filtering and centrifuging, the precipitate was collected and vacuum-dried, giving desired polysaccharides (13 g). The content of the polysaccharides was measured by phenolsulfuric method. Result showed that the content of the polysaccharides in the extract may reach 97.54%.

### Example 2

### Stem Cells Migrate Following L. barbarum Consumption

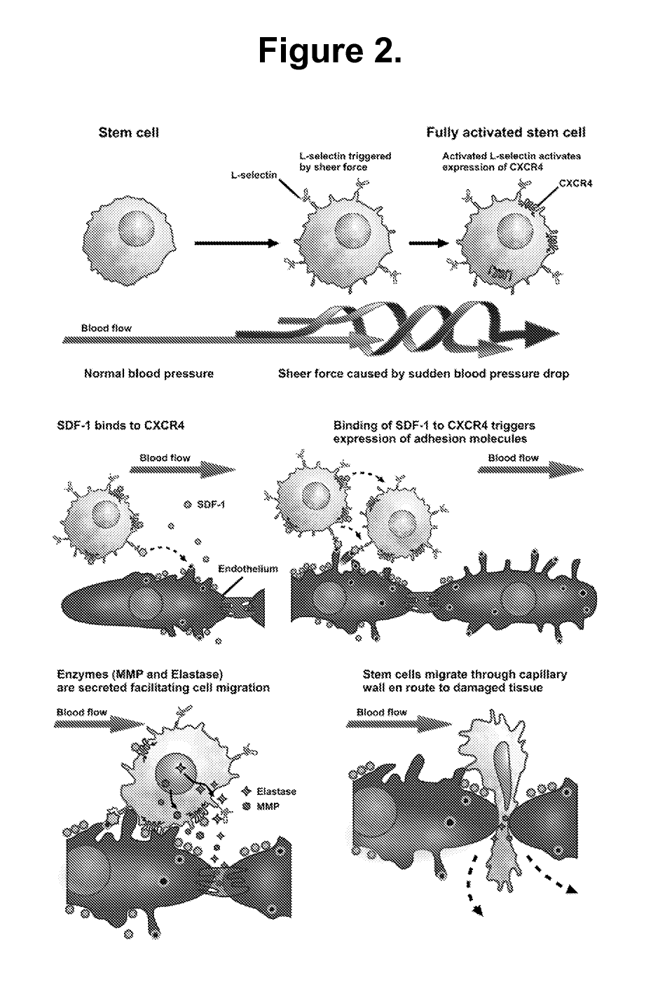

Consumption of Lycium barbarum, or compounds thereof, enhances recruitment and migration of CD34+ stem cells (see FIG. 2 for a diagram of stem cells entering the circulatory system).

Healthy human volunteers were identified, and the proportion of CD34+ cells was evaluated in the peripheral blood (circulating CD34+ cells) of each person prior to consumption of Lycium barbarum and hourly for up to 4 hours after consumption. The volunteers were instructed to limit physical and mental activity for a time before and after consumption of Lycium barbarum.

Each person was provided 5 grams of dried Lycium barbarum or 1 gram of polysaccharide extracted from Lycium barbarum. Red blood cells in whole blood samples obtained from each volunteer were lysed using FACS lysing solution (Beckton Dickenson, San Jose, Calif.). The remaining cells were washed and stained with monoclonal antibody HPCA-2 conjugated with fluorescein isothiocyanate. Samples were fixed in 1% formalin and analyzed by flow cytometry using a FacsCalibur flow cytometer (Becton Dickenson, San Jose, Calif.) and CellQuest software (Becton Dickenson, San Jose, Calif.).

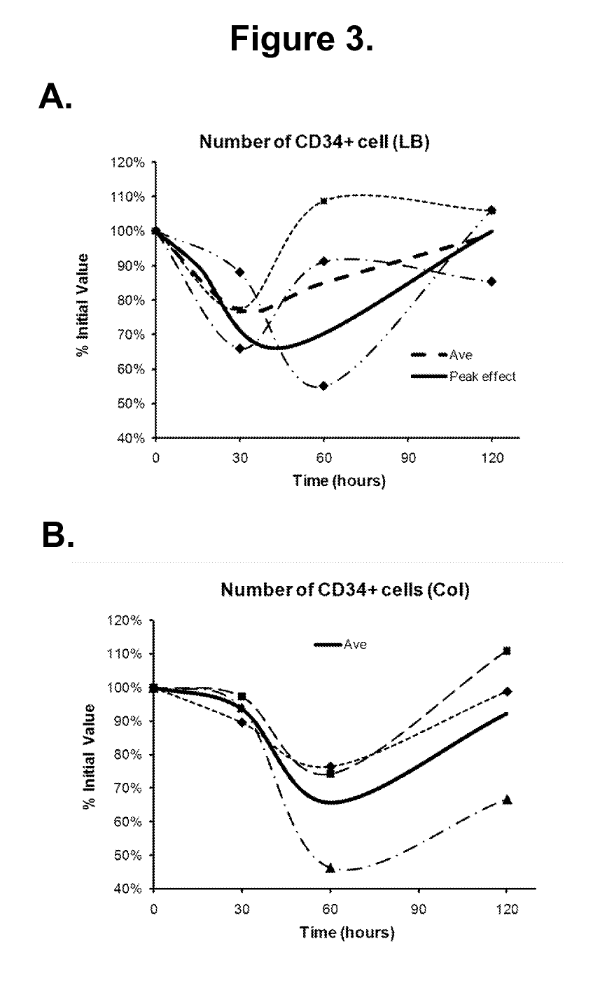

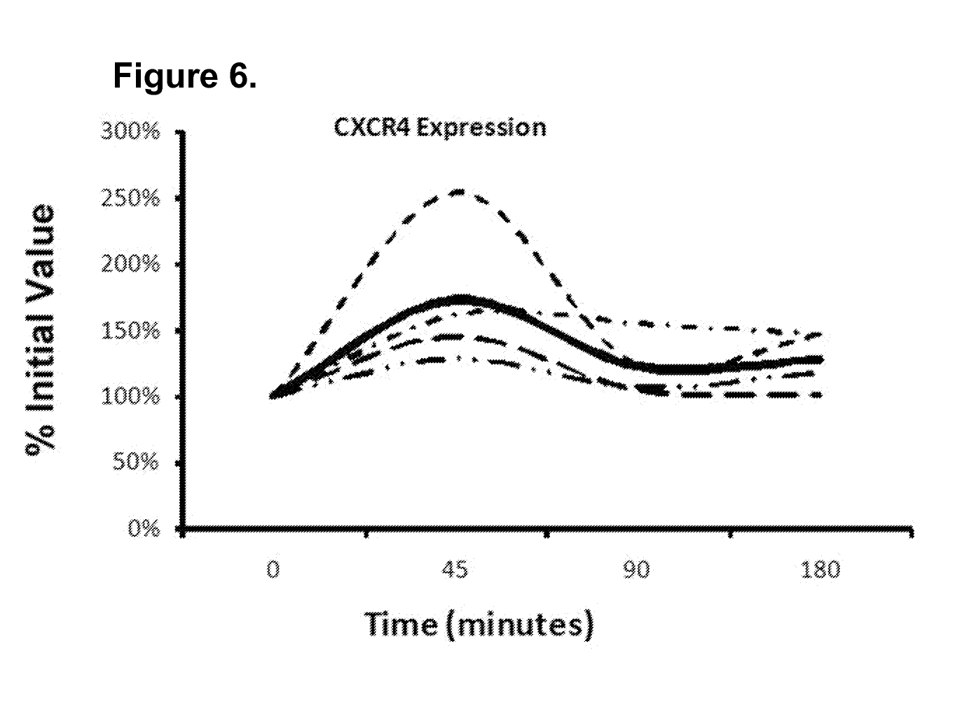

FIG. 3A illustrates that consumption of Lycium barbarum triggered a strong transient decrease in circulating stem cells. Specifically, the X-axis shows the time course of a typical experiment after Lycium barbarum ingestion, expressed as a percentage of the control level. At the time of ingestion, the proportion of circulating CD34+ cells is the same as the control. The peak decrease in circulating CD34+ cells was observed at about 1-2 hours after consumption. At this time point, the number of circulating CD34+ cells was decreased by 30% below the control value. By 4 hours after Lycium barbarum ingestion, the circulating CD34+ cells had returned to the baseline value. The decrease in the number of circulating stem cells was accompanied by an increase in the expression of CXCR4 on the membrane of circulating stem cells (FIG. 6).

Therefore, Lycium barbarum (or a biological component of Lycium barbarum) can enhance the migration of endogenous stem cells (e.g. CD34+ cells) from the circulation to tissues. Consumption of Lycium barbarum (or a biological component of Lycium barbarum) triggers the migration CD34+ stem cells (e.g., see FIG. 3), thereby demonstrating the efficacy of Lycium barbarum as a migration agent.

### Example 3

### Stem Cells Migrate Following Colostrum Consumption

As in Example 2, and with reference to FIG. 3B, administration of colostrum results in stem cell migration.

### Example 4

### Stem Cells Migrate Following Mushroom Consumption

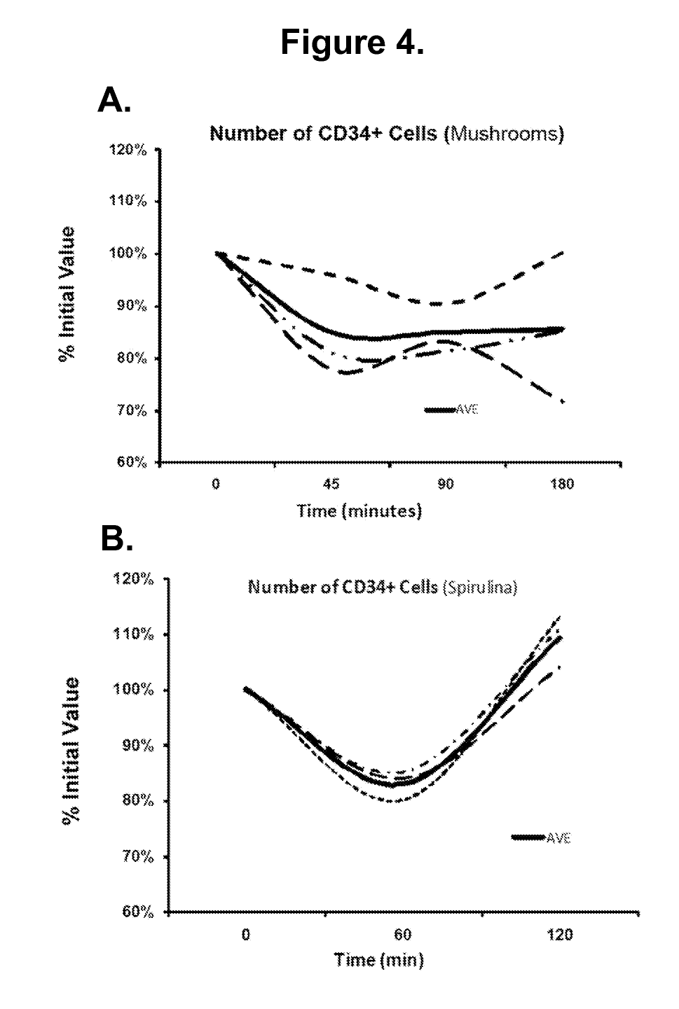

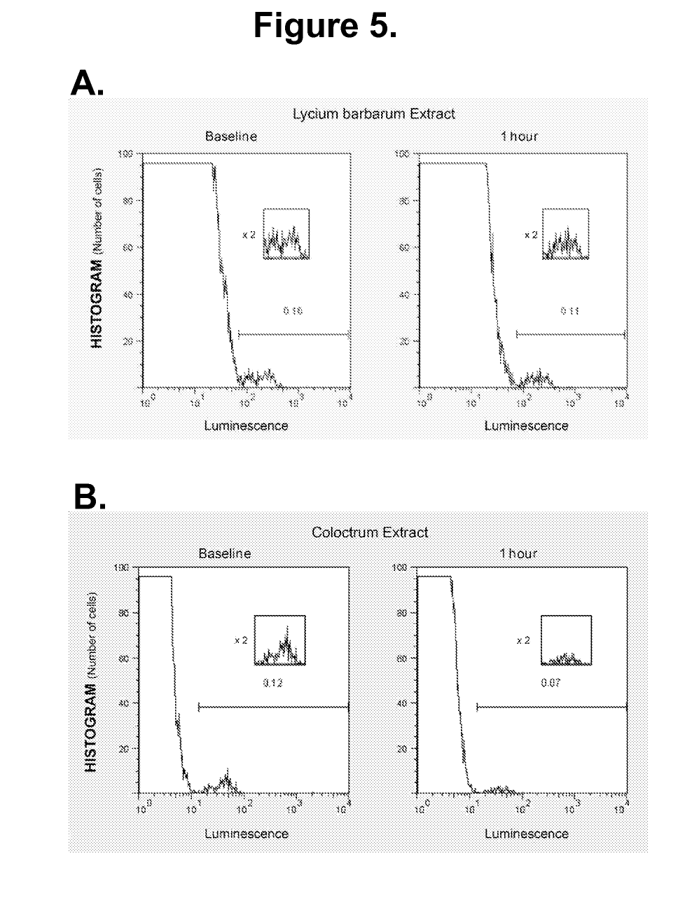

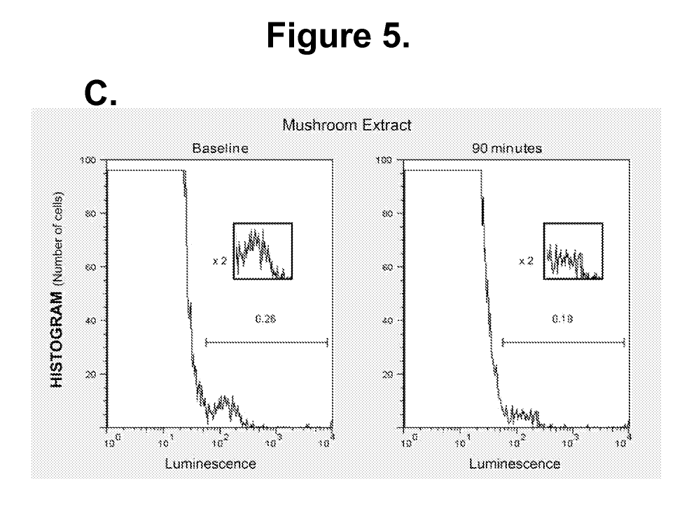

As in Example 2, and with reference to FIG. 4, administration of a polysaccharide rich fraction of mushroom (Cordyceps sinensis, Ganoderma lucidum, Hericium erinaceus) results in stem cell migration.

### Example 5

### Stem Cells Migrate Following Fucoidan or Spirulina Consumption

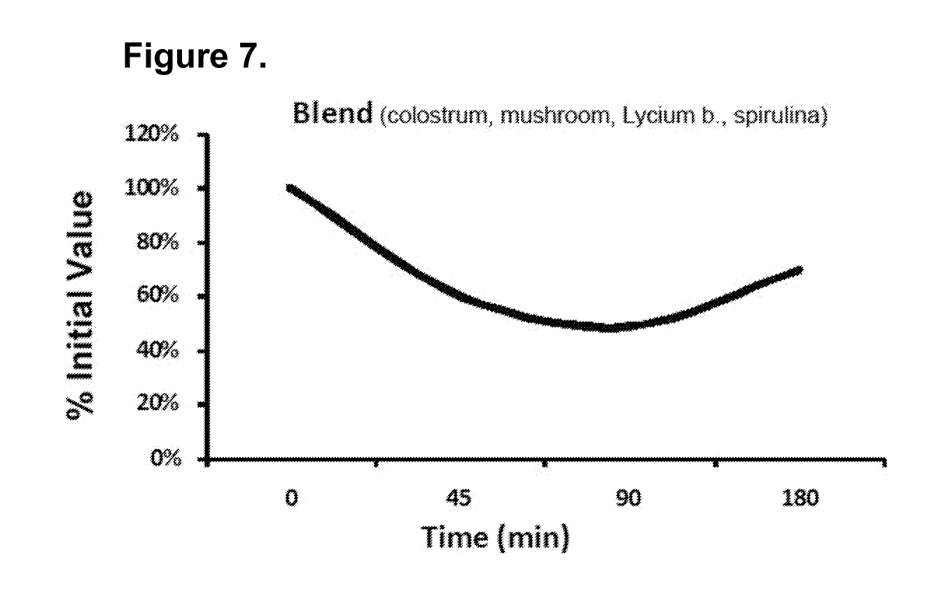

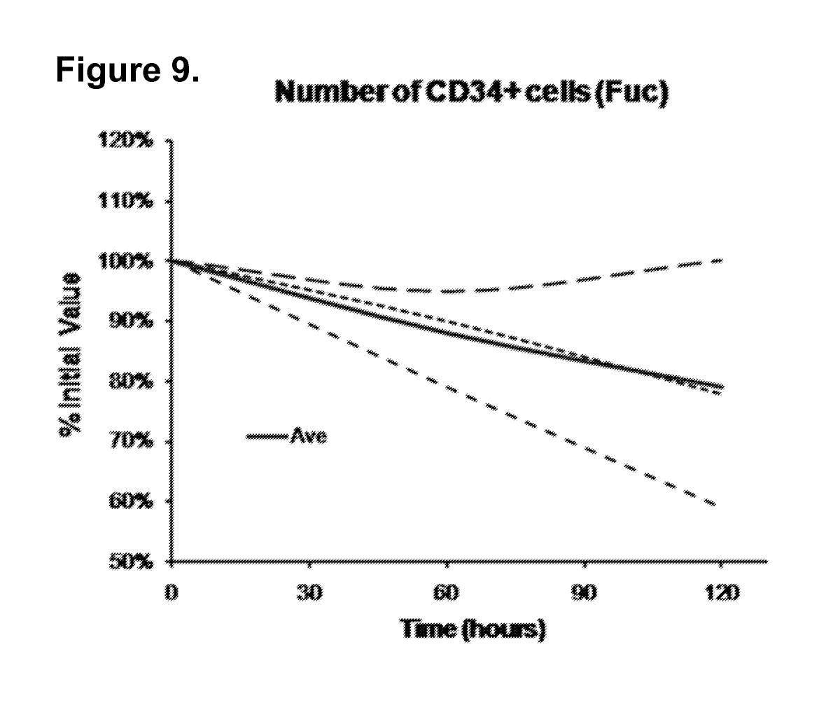

As in Example 2, administration of fucoidan from algae seaweeds such as Chordaria cladosiphon promotes certain beneficial results that may ultimately, albeit indirectly, assist with stem cell migration. For example, consumption of fucoidan from Chordaria cladosiphon resulted in a decrease in the number of circulating CD34+HSCs (FIG. 9), suggesting an effective role in supporting stem cell migration. As in Example 2, administration of spirulina results in stem cell migration (FIG. 4B), and administration of spirulina with Lycium barbarum, colostrum and mushrooms also results in stem cell migration (FIG. 7).

### Example 6

### Stem Cells Migrate Following Consumption of a Blend of LB, Colostrum, Spirulina and Mushroom

Compositions including the following components listed in Table 1 are provided to mammalian subjects. Administration of these compositions results in stem cell migration.

TABLE 1

| | Compo- | Compo- | Compo- | Compo- |
|---|---|---|---|---|
| | sition 1 | sition 2 | sition 3 | sition 4 |
| | mg/dose | mg/dose | mg/dose | mg/dose |
| Lycium barbarum (Goji extract) | 500 | 1,000 | 1,500 | 2,000 |
| Colostrum (Fractionated) | 75 | 150 | 225 | 300 |
| Spirulina | 75 | 150 | 225 | 300 |
| Mushroom 6.255 | 250 | 500 | 750 | 1,000 |
| Hericium erinaceus | 83 | 166 | 249 | 332 |
| Ganoderma lucidum | 83 | 166 | 249 | 332 |
| Cordyceps sinensis | 83 | 166 | 249 | 332 |

### Example 7

### Stem Cells from Bone Marrow Populate Multiple Distant Tissues

A murine model is chosen to evaluate how a mixture of LB, colostrum and mushroom can stimulate stem cell migration into tissues, and therefore populate and repair distant tissues of the body.

Male mice are selected as bone marrow donor animals, while all recipient mice are females. Female recipients are sub-lethally irradiated prior to injection of GFP+ male bone marrow cells into their tail veins. Two groups of mice are evaluated. The first group of 20 animals are sub-lethally irradiated, injected with bone marrow, and put on normal feed. The second group of 20 animals are also sub-lethally irradiated, receive male bone marrow, and are fed a diet of normal feed plus a mixture of LB, colostrum and mushroom. Incorporation of GFP+ cells is examined in the brain, heart muscle, muscles, liver, pancreas, sections of small intestine, and lung tissue

These data document the extent to which a diet containing a mixture of LB, colostrum and mushroom promotes the homing and migration of bone marrow stem cells to various tissues.

### Example 8

### Increased Stem Cell Repopulation of Traumatized Tissue

A murine model is chosen to evaluate how a mixture of LB, colostrum and mushroom can stimulate stem cell migration into tissues, and therefore populate and repair distant tissues of the body.

Male mice are selected as bone marrow donor animals, while all recipient mice are females. Female recipients are sub-lethally irradiated prior to injection of GFP+ male bone marrow cells into their tail veins. Two groups of mice are evaluated. The first group of 20 animals are sub-lethally irradiated, injected with bone marrow, and put on normal feed. The second group of 20 animals are also sub-lethally irradiated, receive male bone marrow, and are fed a diet of normal feed plus a mixture of LB, colostrum and mushroom.

After bone marrow transplant and a few days prior to the initiation of the feeding trial, animals are subjected to an injury such as injection of cardiotoxin in the tibialis muscle, triggering of heart attack by ligation of coronary artery, punch of the skin, laser-induced stroke, or other injuries. The recovery of mice in both groups is monitored during 6 weeks using whole body fluorescence imaging. After 6 weeks, the animals are sacrificed and the injured tissue is analyzed to assess the extent of tissue repair. Incorporation of GFP+ cells will also be examined in the brain, heart muscle, muscles, liver, pancreas, sections of small intestine, and lung tissue

These data document the extent to which a diet containing a mixture of LB, colostrum and mushroom promotes the homing and migration of bone marrow stem cells to injured tissues, therefore enhancing the process of tissue repair and healing.

### Example 9

### General Study Design for Fucoidan as a Stem Cell Mobilization Agent

Two consumables were tested in human subjects: fucoidan extracted from Undaria and a placebo. Peripheral venous blood samples were obtained from healthy human volunteers between 20 and 45 years of age upon informed consent. Blood and bone marrow samples were obtained under aseptic conditions and processed immediately. One gram of fucoidan or placebo was given to volunteers with 4-6 oz water. Appearance of the placebo was identical to that of the fucoidan and consisted of tan-dyed, finely ground potato flakes encapsulated in vegetable capsules.

### Example 10

### In Vivo Study Design

The following exclusion criteria were used: under 20 or over 65 years of age, pregnancy, severe asthma and allergies requiring daily medication, any known chronic illness or previous/current venereal disease, frequent recreational drug use, and impaired digestive function (including previous major gastrointestinal surgery). Three volunteers were scheduled on two study days one week apart. Testing was always performed at the same time of the day (8-11 a.m.) to minimize the effect of circadian fluctuations. Due to the interference from stress with the release vs. homing of other types of lymphocytes, effort was taken to minimize any physical and mental stress during testing. In addition, on each study day, volunteers were instructed to complete a questionnaire aimed at determining any exceptional stress related circumstances that might affect the person on that particular study day. Predetermined criteria for exclusion from final analysis included significant lack of sleep and severe anxiety. After completing the questionnaire, volunteers were instructed to remain quiescent for 4 h, comfortably seated in a chair. After the first hour, the baseline blood sample was drawn. Immediately after drawing the baseline sample, a consumable was provided. Blood samples were later drawn 60, 90 and 180 min after ingestion of the consumable. At each time point, 5 ml of blood was drawn into heparin, and 2 ml blood was drawn into EDTA. The blood vials were placed on a rocking plate until use.

### Example 11

### Measurement of Stem Cell Populations Using FACS Sorting

The blood drawn into EDTA was used for obtaining a complete blood count (CBC) with differential, using a Coulter counter (Micro Diff II, Beckman Coulter). All CBCs were performed within an hour of drawing the sample. All CBCs were performed in triplicate. The heparinized blood was used for purification of the PBMC fraction by gradient centrifugation and processed for immunostaining and flow cytometry. The stem cell markers CD34-FITC (clone 8G12, BD BioSciences, San Jose, Calif., USA) and CD133-PE (Miltenyi Biotech, Auburn, Calif., USA) were used for two color immunofluorescence. Staining of all samples with CD34-FITC/CD133-PE was performed in triplicate. IgG1-FITC and IgG1-PE isotype controls (BD BioSciences) were used in parallel samples. Separate, positive control samples for each donor included CD45-FITC and CD14-PE. Stained PBMC were fixed in 1% formalin and acquired by flow cytometry immediately. Files of 200,000 events were collected on each triplicate sample. The percent CD34+CD133-, CD34+CD133+, and the CD34-CD133+ subsets were analyzed separately and were analyzed again after multiplying with the lymphocyte cell counts, as obtained from the average of the triplicate lymphocyte counts obtained by the CBC differential count.

### Example 12

### Increase in CD34+HSCs Circulating in Peripheral Blood Following Oral Administration of Fucoidan from Undaria pinnatifida

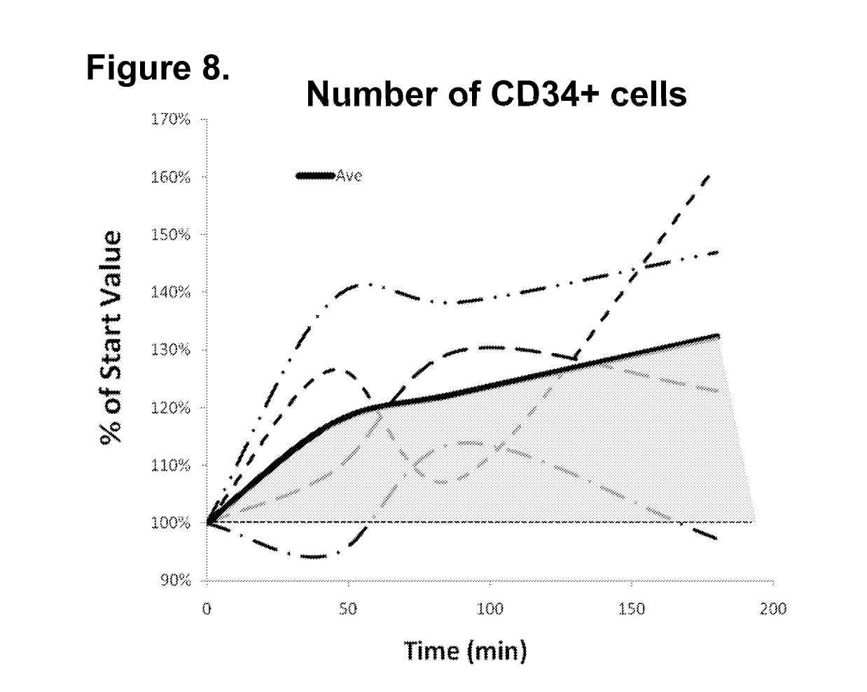

The inventors tested oral administration of fucoidans from several different algae species for their potential to effectuate HSC mobilization in the peripheral bloodstream of human subjects. Fucoidan from one species, Undaria pinnatifida, resulted in a significant elevation in the number of circulating CD34+HSCs, with increases of 17%, 23% (P<0.02) and 32% ((P<0.02) occurring at 45, 90 and 180 minute measurement intervals, thereby demonstrating efficacy as a releasing agent. (FIG. 8) To the best of the inventors' knowledge, this is the most significant increase reported in the literature and further, is a notable improvement over the previously reported 12% increase after 14 days in Irimeh et al., which also tested oral administration of fucoidan from Undaria pinnatifida. Importantly, Irimeh et al. reported 3 gram of fucoidan administered daily, whereas the inventors achieved improved results using a 250 mg dosage regime. This highlights an important role for applying a specific dosage when orally administering fucoidan to promote release and circulation of CD34+HSCs. Furthermore, a lower dosage may permit longer-term patient use, such as routine daily administration, whereas higher dosages may not be compatible with repeated and/or routine use.

### Example 13

### Decrease in CD34+HSCs Circulating in Peripheral Blood Following Oral Administration of Fucoidan from Chordaria cladosiphon

Extending these observations, the inventors discovered that fucoidan from several other algae species, including Chordaria cladosiphon, failed to elevate the circulating number of CD34+HSCs in human subjects (FIG. 9). Despite application of several dosage regimes, including the effective 250 mg dosage of fucoidan from Undaria pinnatifida as described above, fucoidan from Chordaria cladosiphon resulted in a decrease in the number of circulating CD34+HSCs, probably consequent to an increase in CXCR4 expression on the surface of circulating HSCs. These results reflect the complex interplay between the exact source of fucoidan and identifying an effective therapeutic dose. Consumption of 250 mg of this fucoidan from Chordaria cladosiphon gave an average decrease in the number of circulating stem cells (FIG. 3) using the same fucoidan preparation methods and administered under the same conditions in volunteers, thereby demonstrating an effective role in supporting migration of stem cells.

These results are consistent with earlier reports that fucoidan from different sources diverge in structure-activity relationships. Fucoidan fractions from A. nodosum and Pelvetia canculata have been reported to possess anti-coagulant activity through the tri-sulfated disaccharide heparin-like motif involved in HSC mobilization. Particularly notable was the report that sulfation patterns correlated with their anticoagulant activities. A similar molecule from the family of galactans, 3-linked, regularly 2-O-sulfated galactan, possesses anticoagulant activity not found in a corresponding 3-linked, regularly 2-O-sulfated fucan. These reports about anti-coagulant activity and the inventors' observations about HSC mobilization clearly demonstrate that the structure-activity relationships of sulfated fucans, including fucoidan, is not the result from generic features, such as charge density from the presence or absence of certain chemical groups. Instead, biological activity depends critically on the exact structure of the polysaccharide. Necessarily, the different structural fucoidans from distinct species of algae is expected to provide a complex range of efficacies for various therapeutic applications, including HSC mobilization. As described above, this will also require establishing effective therapeutic doses, which may vary when using fucoidans from different species.

### Example 14

### Stem Cells Mobilize Following Consumption of Polygonum multiflorum Alone, or when Included in a Blend of Polygonum multiflorum, Lycium Barbarum, Fucoidan, colostrum, Spirulina and Mushroom

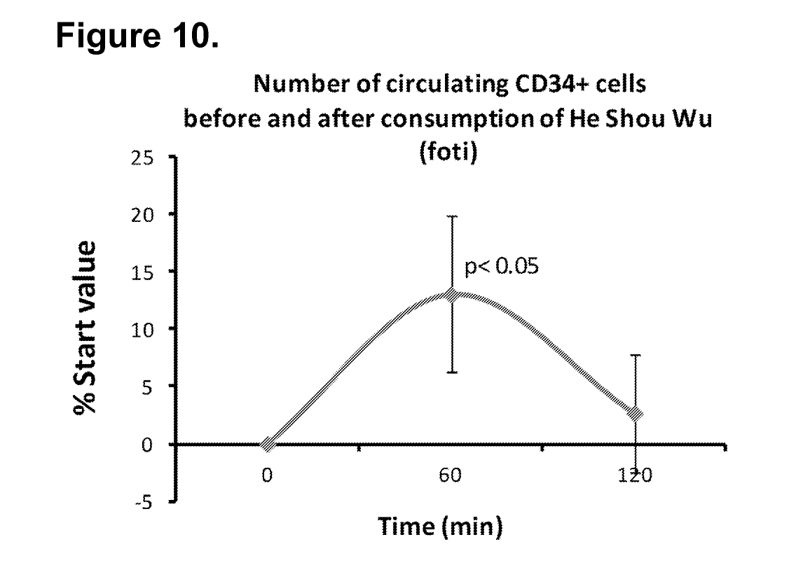

Polygonum multiflorum was shown to trigger a modest increase in the number of circulating stem cells by 13±6% (n=7) (p<0.05). The increase exceeded 25% in 2 or the participants. The results are shown in FIG. 10.

### Example 15

### Increase in CD34+HSCs Circulating in Peripheral Blood Following Oral Administration of Aloe Macroclada

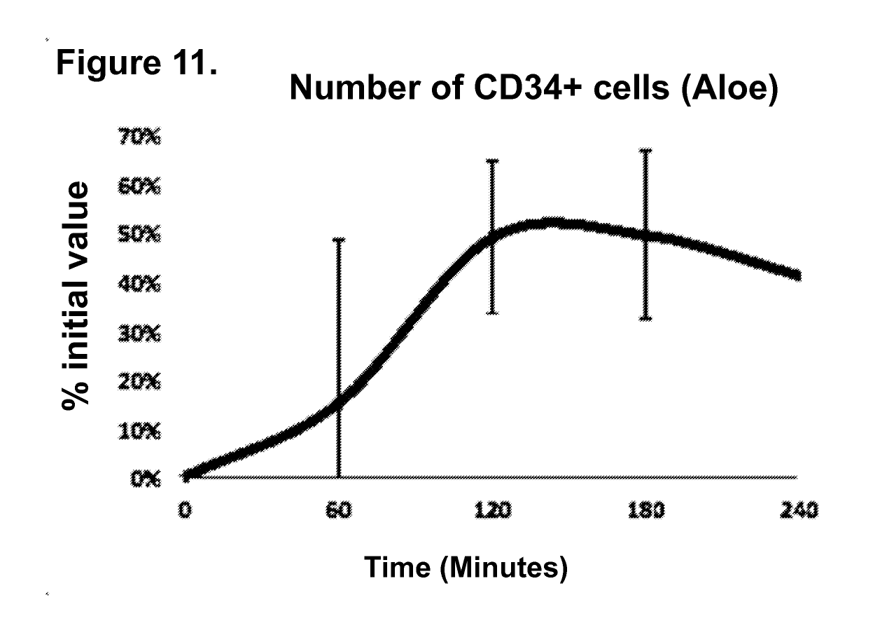

The inventors further tested oral administration of Aloe macroclada from the Aloe genus for potential to enhance HSC mobilization in the peripheral bloodstream of human subjects. As shown in FIG. 11, the levels of stem cells were subsequently measured at 60, 120, 180 and 240 minutes as shown, with a rapid increasing rate of over 60 to 120 minute time points, sustained through subsequent measurements at 180 and 240 minutes.

### Example 16

### Comparison of Aloe macroclada Extract Efficacy and Indigenous Pellets

After the initial documentation of the effect of A. macroclada pellets, handmade by indigenous people of Madagascar, on bone marrow stem cell mobilization, the Inventors tested various parts of the plant for an effect on stem cell mobilization.

Considering that the indigenous pellets are made essentially of a crude preparation of residual sap and plant ash, prepared via burning of plant material, possibly leading to destruction of therapeutically effective components, the Inventors prepared an improved composition plant sap and gel and tested two doses of this blend in human subjects, 250 mg and 750 mg, against the initial mount of 340 mg of pellets.

### Example 17

### Aloe macroclada Enhances Stem Cell Trafficking Across a Variety of Stem Cell Types

Interestingly, despite the crude preparation techniques, both indigenous pellets of A. macroclada as well as the Inventors' devised preparation of a blend of sap and gel showed possible effects on the mobilization of 4 types of stem cells, namely CD45^{dim} CD34⁺, CD34⁺, CD34⁺ KDR⁻, and CD45⁻ CD31⁺ KDR⁺, although the Inventors improved composition consistently triggered a significant increase across all these stem cell types.

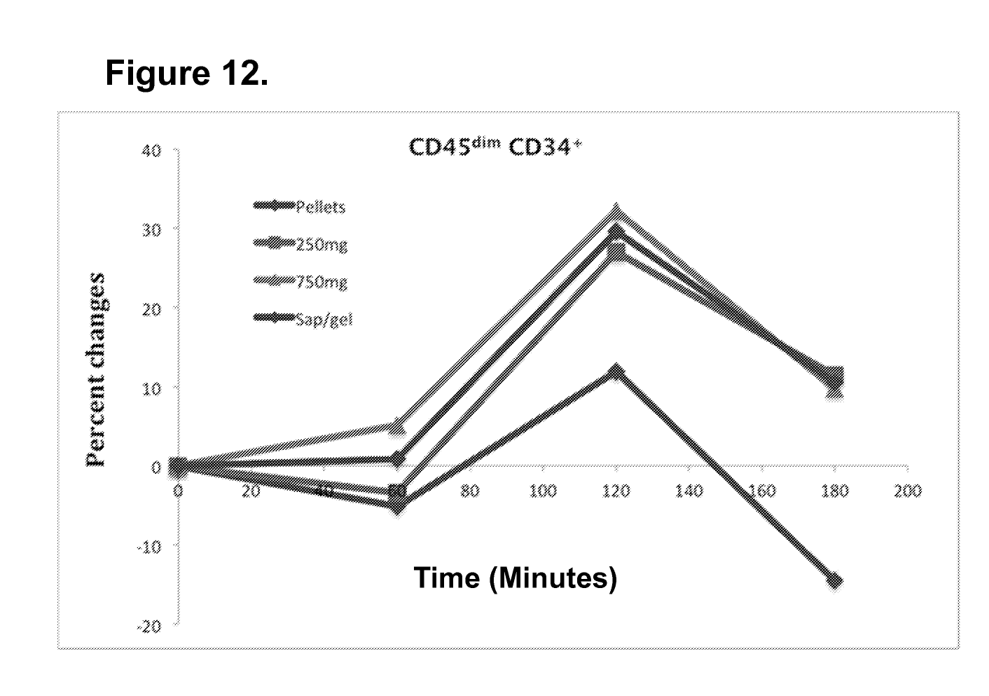

As shown in FIG. 12, indigenous pellets (340 mg) did not have any effect on CD45^{dim} CD34⁺ cells. However, 250 mg and 750 mg of sap/gel triggered an increase in the number of circulating CD45^{dim} CD34⁺ cells that reached 27% and 32% at 120 minutes, though the effect did not reach significance. Results seen with 250 mg and 750 mg of the sap/gel did not show any significant difference and were therefore pooled together. When pooled the data with the sap/gel (n=8) revealed a 29.6% increase (p<0.02) in the number of circulating CD45^{dim} CD34⁺ cells at 120 minutes.

### Example 18

### Variable Effects of Aloe macroclada Preparation Dosages on CD34⁺ Cell Type

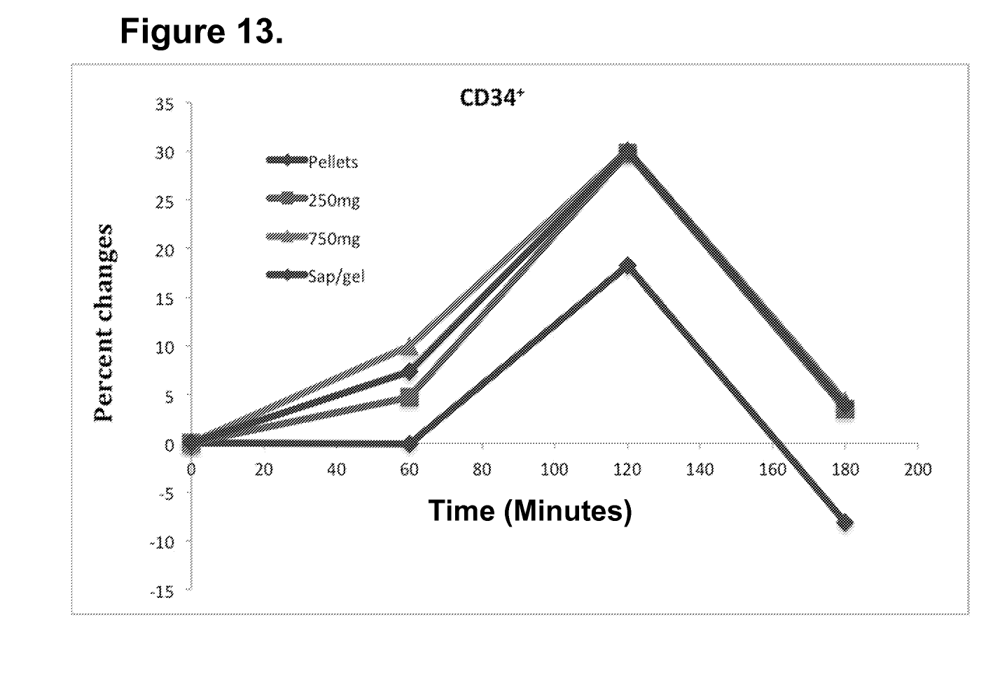

As shown in FIG. 13, indigenous pellets (340 mg) triggered an 18% increase in the number of circulating CD34⁺ cells, though this did not reach significance. Doses of 250 mg and 750 mg of sap/gel triggered an increase in the number of circulating CD34⁺ cells that reached 29.8% and 32% at 120 minutes. However, only the effect seen with 250 mg reached significance (p<0.04).

Results seen with 250 mg and 750 mg of the sap/gel did not show any significant difference and were therefore pooled together. Data with the sap/gel (n=8) revealed a 29.9% increase (p<0.001) in the number of circulating CD34⁺ cells at 120 minutes.

### Example 19

### Variable Effects of Aloe macroclada Preparations Dosages on CD34⁺ KDR⁻ Cell Type

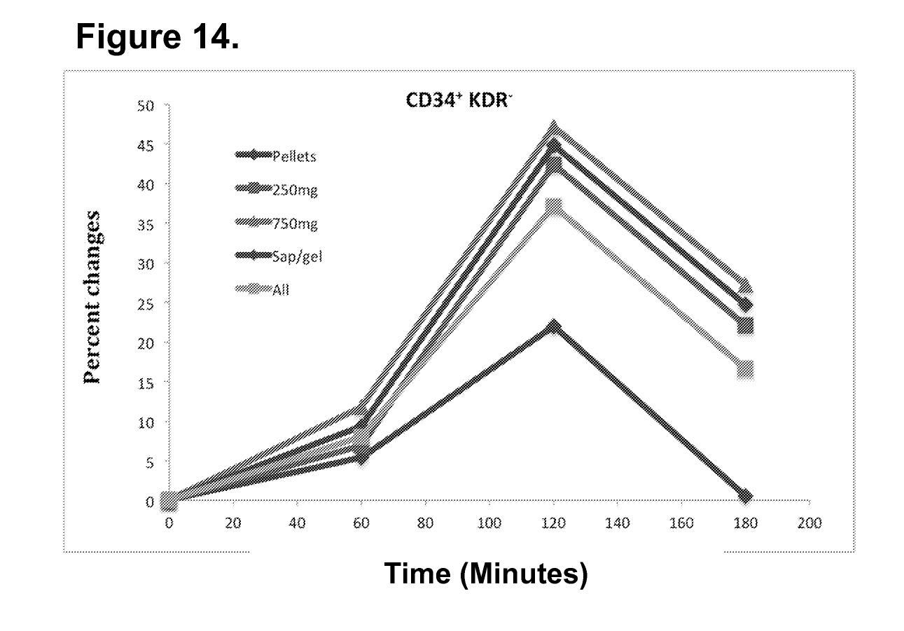

As shown in FIG. 14, indigenous pellets (340 mg) triggered a 21.9% increase in the number of circulating CD34⁺ KDR⁻ cells at 120 minutes (p<0.03). Doses of 250 mg triggered an increase in the number of circulating CD34⁺ KDR⁻ cells of 42.4% at 120 and 22% at 180 minutes, though the effect did not reach significance. Doses of 750 mg triggered an increase in the number of circulating CD34⁺ KDR⁻ cells of 47.2% at 120 and 27.2% at 180 minutes, though the effect also did not reach significance.

Results seen with 250 mg and 750 mg of the sap/gel did not show any significant difference and were therefore pooled together. All data with the sap/gel (n=8) revealed a significant 44.8% increase in the number of circulating CD34⁺ cells at 120 minutes (p<0.01) and 24.7% at 180 minutes (p<0.02).

### Example 20

### Variable Effects of Aloe macroclada Preparations Dosages on CD45CD34⁺ KDR⁺ Cell Type

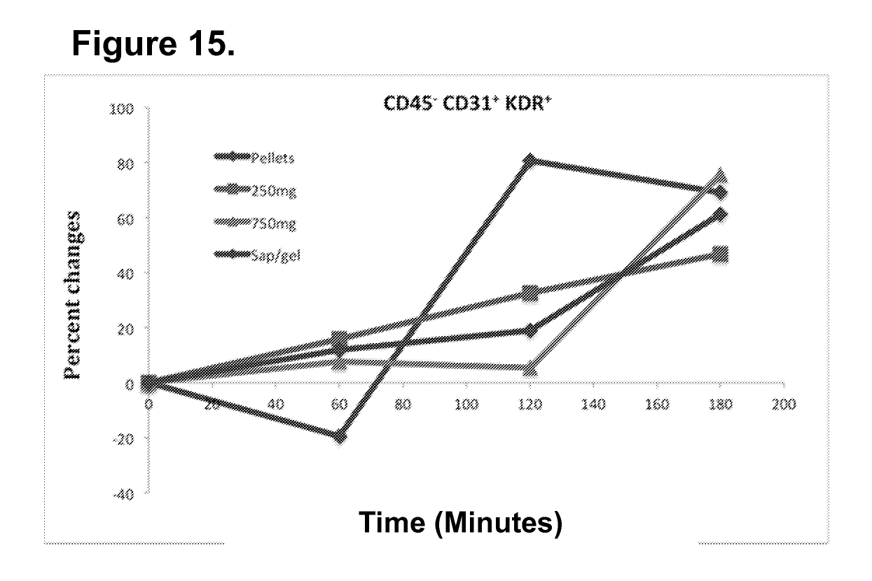

As shown in FIG. 15, indigenous pellets (340 mg) triggered an 80.6% and 69% increase in the number of circulating CD45⁻ CD31⁺ KDR⁺ cells at 120 (p<0.02) and 180 minutes (p<0.03), respectively. Doses of 250 mg triggered an increase in the number of circulating CD45⁻ CD31⁺ KDR⁺ cells of 32.4% and 46.8% at 120 and 180 minutes, respectively, though only the effect at 180 minutes reach significance (p<0.003). Doses of 750 mg triggered a significant increase in the number of circulating CD45⁻ CD31⁺ KDR⁺ cells of 75.4% at 180 (p<0.02). Results seen with 250 mg and 750 mg of the sap/gel did not show any significant difference and were therefore pooled together. All data with the sap/gel (n=8) revealed a 61.1% increase (p<0.0004) in the number of circulating CD34⁺ cells at 180 minutes.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the sources of stem cell mobilization agents, the methods of preparing, isolating, or purifying stem cell mobilization agents, analogs and derivatives thereof, methods of treating various disease and/or conditions using stem cell mobilization agents, analogs and derivatives thereof, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of increasing stem cell mobilization in a subject, comprising:
providing a mobilization agent capable of increasing stem cell mobilization; and
administering a quantity of the mobilization agent to the subject in an amount sufficient to increase stem cell mobilization in the subject.

2. The method of claim 1, wherein the mobilization agent is a composition comprising one or more of the following components selected from the group consisting of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof.

3. The method of claim 1, wherein the mobilization agent comprises Aloe.

4. The method of claim 3, wherein the Aloe is Aloe macroclada.

5. The method of claim 1, wherein the stem cell comprises a bone marrow-derived stem cell (BMSC).

6. The method of claim 1, wherein the stem cell comprises a hematopoietic stem cell (HSC).

7. The method of claim 1, wherein administering the quantity comprises oral administration.

8. The method of claim 7, wherein oral administration is more than once a day.

9. The method of claim 7, wherein oral administration is daily.

10. The method of claim 7, wherein the oral administration comprises use of a capsule.

11. The method of claim 7, wherein the capsule comprises a quantity of about 50, 100, 150, 200, 250 mg or less of the one or mobilization agents.

12. The method of claim 7, wherein the capsule comprises a quantity of about 250, 500, 750, or 1000 mg or less of the one or more mobilization agents.

13. The method of claim 12, comprising 750 mg or less of Aloe macroclada.

14. A pharmaceutical composition comprising:
one or more of the following components selected from the group consisting of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof; and
a pharmaceutically acceptable carrier.

15. The pharmaceutical composition of claim 14, wherein the pharmaceutical composition comprises Aloe.

16. The pharmaceutical composition of claim 15, wherein the Aloe is Aloe macroclada.

17. The pharmaceutical composition of claim 14, wherein comprising a quantity of about 50, 100, 150, 200, 250 mg or less of the one or more components.

18. The pharmaceutical composition of claim 14, comprising a quantity of about 250, 500, 750, or 1000 mg or less of the one or more components.

19. The pharmaceutical composition of claim 18, comprising 750 mg or less of Aloe macroclada.

20. The pharmaceutical composition of claim 19, comprising 750 mg or less of Aloe macroclada and 1000 mg or less of one or more of the following components selected from the group consisting of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof.
